(19) United States
(12) Patent Application Publication
(10) Pub. No.: US 2022/0304615 A1
(43) Pub. Date: Sep. 29, 2022

(54) MEASUREMENT SYSTEM, MEASUREMENT APPARATUS, AND INFORMATION PROCESSING APPARATUS

(71) Applicants: Hironobu MIFUNE, Miyagi (JP); Takanobu OSAKA, Hokkaido (JP)

(72) Inventors: Hironobu MIFUNE, Miyagi (JP); Takanobu OSAKA, Hokkaido (JP)

(21) Appl. No.: 17/700,508

(22) Filed: Mar. 22, 2022

(30) Foreign Application Priority Data
Mar. 23, 2021 (JP) 2021-048803

(51) Int. Cl. A61B 5/384; A61B 5/291; A61B 5/256

(52) U.S. Cl. CPC A61B 5/384; A61B 5/291; A61B 5/256

(57) ABSTRACT

A measurement system includes: a mounting member configured to be mounted on a measurement target portion of a living body and including a plurality of holding units configured to hold a plurality of magnetic sensors such that the plurality of magnetic sensors face the measurement target portion; an image capturing unit configured to capture images of markers arranged in arrangement regions of the plurality of holding units of the mounting member mounted on the measurement target portion, in the measurement target portion; a moving mechanism unit configured to move the image capturing unit relative to the measurement target portion on which the mounting member is mounted; and a derivation unit configured to derive three-dimensional position information on the plurality of magnetic sensors held by the plurality of holding units relative to the measurement target portion, based on the captured images of the markers.

### CROSS-REFERENCE TO RELATED APPLICATIONS

The present application claims priority under 35 U.S.C. § 119 to Japanese Patent Application No. 2021-048803, filed on Mar. 23, 2021. The contents of which are incorporated herein by reference in their entirety.

### BACKGROUND OF THE INVENTION

### 1. Field of the Invention

The present invention relates to a measurement system, a measurement apparatus, and an information processing apparatus.

### 2. Description of the Related Art

A technique for detecting magnetic signals generated from a living body is known. For example, a holding member, such as a helmet, that holds a plurality of magnetic sensors is mounted on a measurement target portion, such as a head portion of a living body, and a magnetic signal at each of positions of the magnetic sensors is measured. Further, a technique for performing signal processing on the magnetic signal at each of the positions of the magnetic sensors and outputting a measurement image that represents the position at which the magnetic signal is generated in the measurement target portion has been known. To detect the position at which the magnetic signal is generated on the living body, it is necessary to identify three-dimensional position information on each of the magnetic sensors with respect to the measurement target portion of the living body.

As a technique for identifying the three-dimensional position information on the magnetic sensors, a technique for identifying the three-dimensional positions of the magnetic sensors by using captured images obtained by a plurality of stereo cameras is disclosed. Further, a technique for providing three-dimensional shape data of a target object viewed from a specific viewpoint by using a camera held by a photographer, and a technique for providing the three-dimensional position information on the magnetic sensors are disclosed.

However, in the method of using the captured images obtained by the stereo cameras, in some cases, it may be difficult to capture images of the magnetic sensors in an environment in which visual fields of the stereo cameras are not fully ensured. Further, in the method of using the camera held by the photographer, in some cases, it may be difficult to fully capture images of the magnetic sensors. Therefore, in the conventional technologies, it is difficult to obtain the three-dimensional position information on the magnetic sensors with high accuracy. In other words, in the conventional technologies, it is difficult to obtain the three-dimensional position information with high accuracy as the measurement positions of the magnetic signals in the measurement target portion of the living body.

Conventional techniques are described in Japanese Patent No. 3907753, Japanese Patent No. 5712640, Japanese Patent No. 5571128, Japanese Unexamined Patent Application Publication No. 2019-164109, “Recording brain activities in unshielded Earth's field with optically pumped atomic

magnetometers”, Rui Zhang, Wei Xiao, Yudong Ding, Yulong Feng, Xiang Peng, Liang Shen, Chenxi Sun, Teng Wu, Yulong Wu, Yucheng Yang, Zhaoyu Zheng, Xiangzhi Zhang, Jingbiao Chen, Hong Guo, Peking University, Science Advances, Vol. 6, No. 24, 10 Jun. 2020, eaba8792, and “Multi-Channel Whole-Head OPM-MEG: Helmet Design and a Comparison with a Conventional System” Ryan M. Hill, Elena Boto, Molly Rea, Niall Holmes, James Leggett, Laurence A. Coles, Manolis Papastavrou, Sarah Everton, Benjamin A. E. Hunt, Dominic Sims, James Osborne, Vishal Shah, Richard Bowtell, Matthew J. Brookes, University of Nottingham, neuroimage. 2020. 116995

### SUMMARY OF THE INVENTION

According to an aspect of the present invention, a measurement system includes a mounting member, an image capturing unit, a moving mechanism unit, and a derivation unit. The mounting member is configured to be mounted on a measurement target portion of a living body and including a plurality of holding units configured to hold a plurality of magnetic sensors such that the plurality of magnetic sensors face the measurement target portion. The image capturing unit is configured to capture images of markers arranged in arrangement regions of the plurality of holding units of the mounting member mounted on the measurement target portion, in the measurement target portion. The moving mechanism unit is configured to move the image capturing unit relative to the measurement target portion on which the mounting member is mounted. The derivation unit is configured to derive three-dimensional position information on the plurality of magnetic sensors held by the plurality of holding units relative to the measurement target portion, based on the captured images of the markers.

### BRIEF DESCRIPTION OF THE DRAWINGS

FIG. 1 is a schematic diagram of a measurement system according to an embodiment;

FIG. 2A is a schematic diagram of a mounting member;

FIG. 2B is a schematic diagram of a holding unit;

FIG. 3 is a schematic diagram illustrating an example of a magnetic sensor;

FIG. 4 is an enlarged schematic diagram of a moving mechanism unit;

FIG. 5 is a schematic diagram illustrating examples of captured images;

FIG. 6 is an exemplary functional block diagram of the measurement system;

FIG. 7 is a flowchart illustrating an example of the flow of information processing;

FIG. 8 is a schematic diagram of a measurement system according to another embodiment;

FIG. 9 is an enlarged schematic diagram of a moving mechanism unit;

FIG. 10 is an exemplary functional block diagram of the measurement system;

FIG. 11A is a diagram for explaining an example of a movement pattern of an image capturing unit;

FIG. 11B is a diagram for explaining an example of a movement pattern of the image capturing unit;

FIG. 11C is a diagram for explaining an example of a movement pattern of the image capturing unit;

FIG. 12 is a flowchart illustrating an example of the flow of information processing;

FIG. 13 is a schematic diagram illustrating an example of a moving mechanism unit;

FIG. 14 is a schematic diagram illustrating an example of a moving mechanism unit;

FIG. 15 is a schematic diagram illustrating an example of a moving mechanism unit; and

FIG. 16 is a hardware configuration diagram.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to provide a measurement system, a measurement apparatus, and an information processing apparatus that are able to obtain three-dimensional position information with high accuracy as a measurement position of a magnetic signal in a measurement target portion of a living body.

Embodiments of a measurement system, a measurement apparatus, and an information processing apparatus will be described in detail below with reference to the accompanying drawings.

### First Embodiment

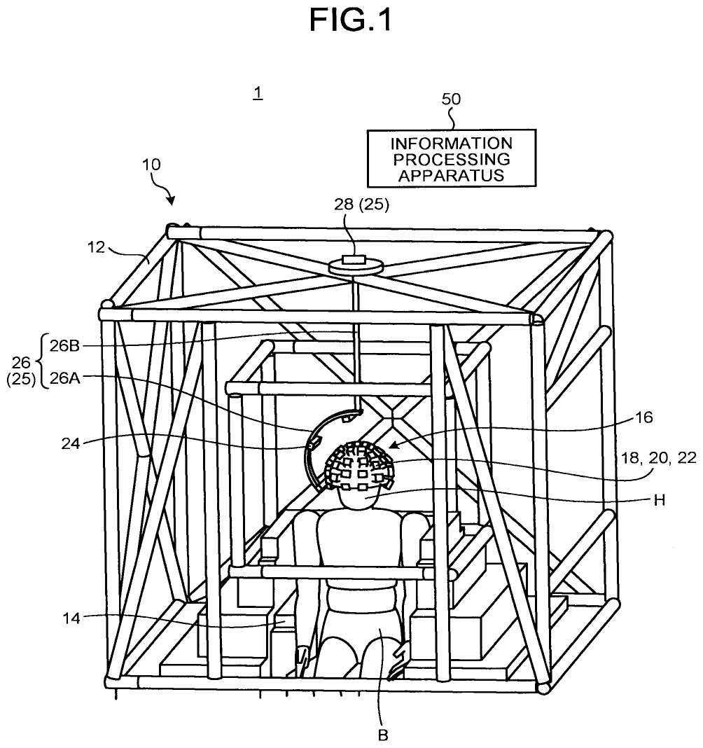

FIG. 1 is a schematic diagram illustrating a measurement system 1 according to a first embodiment. The measurement system 1 includes a measurement apparatus 10 and an information processing apparatus 50. The measurement apparatus 10 and the information processing apparatus 50 are communicably connected to each other.

The measurement apparatus 10 is an apparatus that measures a magnetic signal generated from a measurement target portion of a living body B. The living body B is an object that lives. The living body B is, for example, an animal, such as a human being. In the present embodiment, a case in which the living body B is a human being will be described as an example. The measurement target portion is a portion in which a magnetic signal is to be measured in the living body B. In the present embodiment, a case will be described in which the measurement target portion is a head portion H of the living body B.

The measurement apparatus 10 includes a housing 12, a seat portion 14, a mounting member 16, image capturing units 24, and a moving mechanism unit 25.

The housing 12 is a member for holding or supporting various mechanisms in the measurement apparatus 10. The seat portion 14 is a portion on which the living body B sits. The seat portion 14 is configured as a part of the housing 12, for example. A magnetic signal generated from the head portion H is measured while the living body B is sitting on the seat portion 14.

The mounting member 16 is a portion that is mounted on the head portion H of the living body B. The mounting member 16 includes a plurality of holding units 18. The holding units 18 are members for holding a plurality of magnetic sensors 20 such that the magnetic sensors 20 face the head portion H. Holding the magnetic sensors 20 such that the magnetic sensors 20 face the head portion H means holding the magnetic sensors 20 such that measurement surfaces of the magnetic sensors 20 face the head portion H. In other words, the holding units 18 hold the magnetic sensors 20 such that the magnetic sensors 20 can measure magnetic signals generated from the head portion H. It is preferable that the holding units 18 are configured to be able to hold the magnetic sensors 20 such that the magnetic sensors 20 come into contact with the head portion H. The holding units 18 hold the magnetic sensors 20 in a detachably attachable manner.

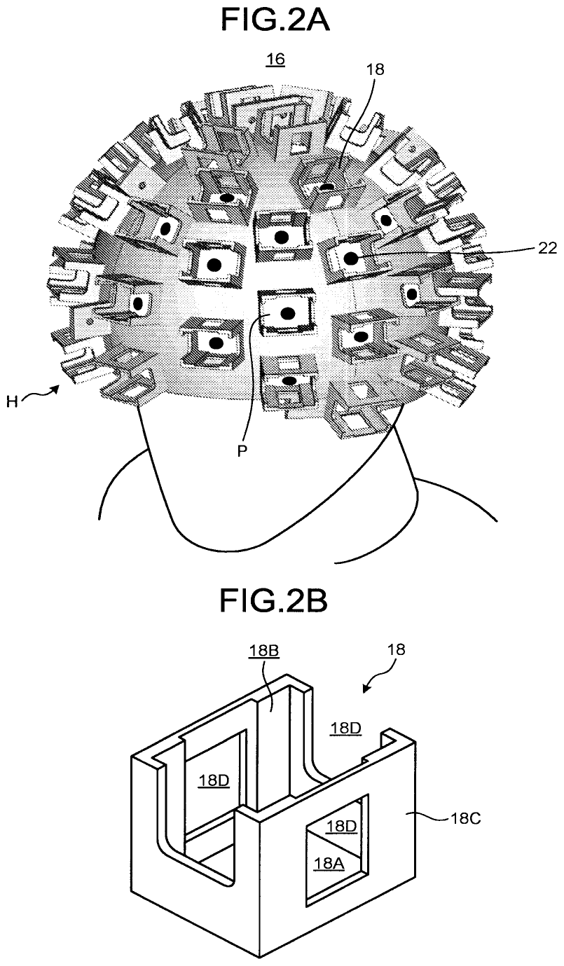

FIG. 2A is a schematic diagram of the mounting member 16. FIG. 2B is a schematic diagram of one of the holding units 18.

The mounting member 16 has a shape that conforms to an outer shape of the head portion H that is one example of the measurement target portion. In the present embodiment, the mounting member 16 has a semispherical shape that conforms to the outer shape of the head portion H that has a spherical shape. In the present embodiment, the mounting member 16 is configured as a helmet that is worn on the head portion H.

The mounting member 16 may be formed in any shape as long as the shape conforms to the outer shape of the head portion H. A material of the mounting member 16 is not specifically limited. For example, the mounting member 16 may be formed of a hard material member that is made by a three-dimensional (3D) printer or the like, a soft material member that is made with fabric, resin, or the like, etc. The mounting member 16 may be made, in advance, with a material corresponding to the living body B, measurement contents, or the like.

FIG. 2A illustrates one example of the mounting member 16 that is made with a hard material and that is made by a 3D printer or the like. In the mounting member 16, the plurality of holding units 18 for holding the magnetic sensors 20 are arranged at different positions. The holding units 18 need not always be arranged over the entire region of the mounting member 16 as illustrated in FIG. 2A as long as the holding units 18 are arranged on the mounting member 16. In the present embodiment, a case in which the holding units 18 are arranged over the entire region of the mounting member 16 will be described as one example. In this case, it is possible to measure magnetic signals in the entire region of the head portion H on which the mounting member 16 is mounted or magnetic signals at arbitrary positions in the entire region.

FIG. 2A and FIG. 2B illustrate one example of the mounting member 16 in which the magnetic sensors 20 are not held. In the holding units 18, sides on an inward side of the mounting member 16 are sides that face the head portion H and sides on an outward side of the mounting member 16 are sides on which the magnetic sensors 20 are inserted. The magnetic sensors 20 are inserted in the holding units 18 from the outward side to the inward side of the mounting member 16 and held by the holding units 18, so that the magnetic sensors 20 are arranged so as to come into contact with or come close to a scalp of the head portion H.

Markers 22 are arranged in arrangement regions P of the holding units 18 of the mounting member 16 mounted on the head portion H, in the head portion H. The markers 22 may be arranged directly on the scalp of the head portion H or may be arranged on a cap that is put on the head portion H and that fits the head portion H.

The markers 22 are used to obtain three-dimensional position information on the magnetic sensors 20 that are held by the holding units 18 in the arrangement regions P of the markers 22. The markers 22 are not specifically limited as long as the markers 22 are attachable to the head portion H.

It is not realistic to prepare the mounting member 16 in accordance with the head portion H of each of subjects as examples of the living body B. Therefore, one or the plurality common mounting members 16 are shared by a plurality of subjects. In this case, in some cases, a gap may be generated between an inner side of the mounting member 16 and the scalp of the head portion H because of a difference in a size of the head portion H of the subject and a size of the mounting member 16. Therefore, the markers 22 are arranged in the arrangement regions P that are regions that come into contact with the magnetic sensors 20 in the head portion H, before the magnetic sensors 20 are held by the mounting member 16. Further, the information processing apparatus 50 of the present embodiment uses the positions of the markers 22 as three-dimensional position information on the magnetic sensors 20 (details will be described later).

It is preferable that the markers 22 have cross-sectional shapes that conform to a surface of the head portion H when the markers 22 are arranged on the head portion H. Therefore, as a material and a thickness of each of the markers 22, it is preferable to adopt a material and a thickness that allow deformation along an outer surface of the head portion H when the markers 22 are arranged on the head portion H. Specifically, it is preferable that the markers 22 has a certain thicknesses. Further, any material that can be bonded or fixed to the head portion H is applicable as the material of the markers 22. Furthermore, any material that can easily be removed from the head portion H after completion of image capturing by the image capturing units 24 is applicable as the material of the markers 22.

The markers 22 may have arbitrary shapes in a direction intersecting a thickness direction as long as the shapes have certain sizes that can be captured by the image capturing units 24. For example, the markers 22 may be formed in arbitrary shapes, such as circular shapes or rectangular shapes, which are formed of curved sides or linear side. The markers 22 may have any colors as long as the colors are distinguishable on captured images that are captured by the image capturing units 24.

The markers 22 are arranged in the arrangement regions P of the holding units 18 that hold the magnetic sensors 20 among the plurality of holding units 18 of the mounting member 16. Therefore, the markers 22 may be arranged in the arrangement regions P of all of the holding units 18 provided in the mounting member 16, or may be arranged in the arrangement regions P of some of the holding units 18.

It is preferable that the holding units 18 of the mounting member 16 mounted on the head portion H have shapes such that at least parts of the markers 22 arranged in the arrangement regions P of the holding units 18 on the head portion H are not located in the blind areas when the image capturing units 24 perform image capturing as will be described later. In other words, it is preferable that the mounting member 16 has a shape that prevents the markers 22 from being located in the blind areas by the holding units 18 when the markers 22 are captured from outside of the mounting member 16.

Specifically, in the present embodiment, each of the holding units 18 includes an opening 18A in at least a part of a region that faces the head portion H in the holding unit 18 when the mounting member 16 is mounted on the head portion H. Further, each of the holding units 18 includes openings 18D on side walls 18C that connect the opening 18A located in an inward direction of the mounting member 16 and an opening 18B located in an outward direction of the mounting member 16 in the holding unit 18. Therefore, the mounting member 16 is able to prevent the markers 22 from being shadowed by the holding units 18 and located in the blind areas of the image capturing units 24 when the markers 22 are captured from a camera viewpoint outside the mounting member 16. In other words, the mounting member 16 is able to prevent missing of the markers 22 in captured images that are captured by the image capturing units 24 (to be described later) when image capturing is performed when the mounting member 16 is mounted on the head portion H.

Meanwhile, each of the holding units 18 need not always be formed in the shape as illustrated in FIG. 2B, but may be formed in any shape as long as it is possible to prevent the markers 22 from being located in the blind areas by the holding units 18.

In some cases, there may be a difference between the size of the head portion H and the size of the mounting member 16 depending on a living body B as a subject. In this case, the holding units 18 may further include configurations that can hold the magnetic sensors 20 while the magnetic sensors 20 come into contact with the scalp of the head portion H.

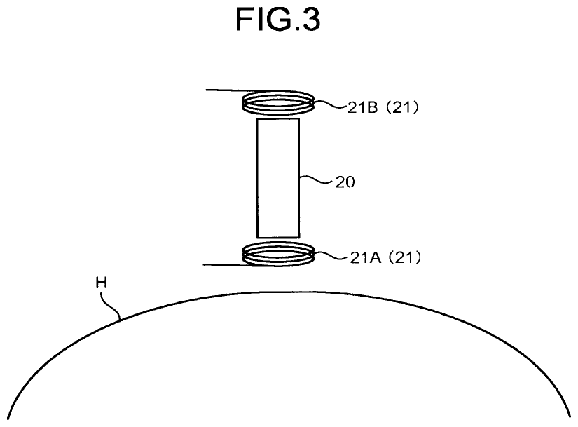

FIG. 3 is a schematic diagram illustrating an example of the magnetic sensors 20. The magnetic sensors 20 are held by the holding units 18 of the mounting member 16 such that the magnetic sensors 20 come into contact with or come close to the head portion H.

Each of the magnetic sensors 20 is, for example, a room-temperature magnetic sensor. The room-temperature magnetic sensor is a magnetic sensor for which a cooling mechanism is not needed. Examples of the room-temperature magnetic sensor include a magneto resistive sensor and an atomic magnetic sensor. Examples of the atomic magnetic sensor include an optically pumped atomic magnetic sensor. Any magnetic sensor may be selected in accordance with a measurement purpose and may be used as the magnetic sensors in the measurement apparatus 10.

In the measurement apparatus 10 of the present embodiment, a plurality of cancel coils 21 are arranged around the magnetic sensors 20. The cancel coils 21 are one example of environmental magnetic field reduction coils for reducing residual magnetic fields. The magneto resistive sensor or the atomic magnetic sensor operates under a residual magnetic field equal to or less than a certain level. Therefore, the cancel coils 21 form magnetic field regions in which the magnetic sensors 20 are operable.

FIG. 3 illustrates a configuration in which a cancel coil 21A is arranged on a side of each of the magnetic sensors 20 facing the head portion H and a cancel coil 21B is arranged on the opposite side. Meanwhile, the number of the cancel coils 21 may be arbitrary as long as the residual magnetic fields at which the magnetic sensors 20 are operable or less can be obtained. Therefore, the number of the cancel coils 21 arranged for each of the magnetic sensors 20 is not limited to two, and may be adjusted in accordance with a usage environment or characteristics of the magnetic sensors 20.

In the information processing apparatus 50 to be described later, values of currents that flow through the cancel coils 21 are optimized in accordance with output of a magnetic field sensor that is used to measure an effect of reduction of environmental magnetism by the cancel coils 21. Through this process, the information processing apparatus 50 adjusts a magnetic field to a desired magnetic field (details will be described later).

Referring back to FIG. 1, explanation is continued. The moving mechanism unit 25 will be described below.

The moving mechanism unit 25 is a moving mechanism that moves the image capturing units 24 relative to the head portion H of the living body B on which the mounting member 16 is mounted. The moving mechanism unit 25 is a mechanical unit that moves the image capturing units 24 such that the image capturing units 24 move around the head portion H.

The image capturing units 24 capture images of the markers 22 arranged in the arrangement regions P of the holding units 18. In the present embodiment, a device that obtains a captured image including distance information is used as the image capturing units 24. The image capturing units 24 are, for example, cameras of a time-of-flight (ToF) system, stereo cameras, or the like. The ToF system is a system to obtain a distance by applying infrared light to a measurement object and calculating a time taken by reflected light to return. The stereo camera is a camera that obtains depth information as the distance information by using an interval between two cameras and disparity information on images obtained by the two respective cameras. A camera that can obtain three-dimensional position information with necessary accuracy may be selected as the image capturing units 24.

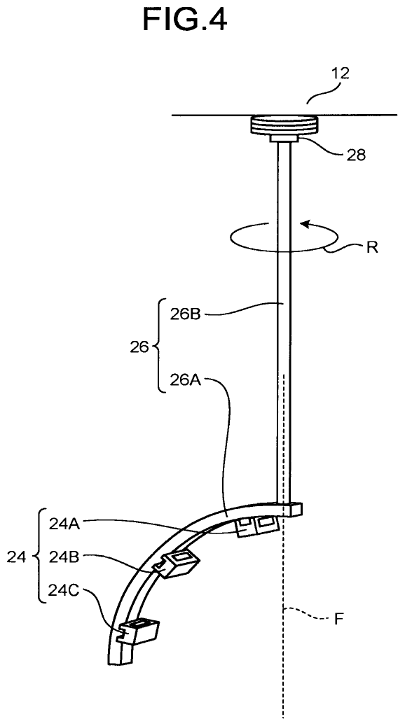

FIG. 4 is an enlarged schematic diagram of the moving mechanism unit 25. The moving mechanism unit 25 includes a support member 26 and a rotation driving unit 28. The support member 26 includes a support member 26A and a shaft 26B.

The support member 26A is a member that supports the image capturing units 24 such that the image capturing units 24 face the head portion H. Supporting the image capturing units 24 such that the image capturing units 24 face the head portion H means supporting the image capturing units 24 such that lens surfaces of the image capturing units 24 face the head portion H. In other words, the support member 26A supports the image capturing units 24 such that the image capturing units 24 are able to capture images of the markers 22 on the head portion H.

In the present embodiment, the support member 26A has a shape that is extended from a top portion of the mounting member 16 along the outer shape of the mounting member 16. The top portion of the mounting member 16 is arranged at a position corresponding to a parietal region of the head portion H when the mounting member 16 is mounted on the head portion H. As described above, in the present embodiment, the mounting member 16 has a semispherical shape. Therefore, in the present embodiment, the support member 26A has a circular arc shape that conforms to the semispherical outer shape of the mounting member 16. It is preferable that a curvature of the support member 26A substantially matches a curvature of the outer shape of the mounting member 16.

The support member 26A supports the plurality of image capturing units 24 at different positions in an extending direction thereof.

Specifically, the support member 26A having the circular arc shape supports the plurality of image capturing units 24 at intervals along the extending direction of the support member 26A. In the present embodiment, the support member 26A supports an image capturing unit 24A, an image capturing unit 24B, and an image capturing unit 24C. An arrangement direction of the plurality of image capturing units 24 matches the extending direction of the support member 26A. Further, the plurality of image capturing units 24 are arranged in advance such that visual fields of the image capturing units 24 located adjacent to each other in the arrangement direction partly overlap with each other. Meanwhile, the number of the image capturing units 24 supported by the support member 26A is not limited to three.

One end portion of the support member 26A in the extending direction is supported by the shaft 26B. One end portion of the support member 26A in a bending direction is adjusted in advance so as to be located at a position corresponding to the parietal region of the head portion H. In other words, one end portion of the support member 26A in the extending direction is adjusted in advance so as to be located at a position corresponding to the top portion of the semispherical mounting member 16 that is mounted on the head portion H.

The shaft 26B is a linear bar-shaped member. One end portion of the shaft 26B is connected to the support member 26A, and the other end portion is connected to the housing 12 via the rotation driving unit 28.

The rotation driving unit 28 rotates the support member 26A about the head portion H as a rotation center. Specifically, in the present embodiment, the rotation driving unit 28 rotates the support member 26A about the shaft 26B as a rotation axis. Therefore, the shaft 26B and the support member 26A are driven to rotate in a direction of arrow R. Further, the rotation driving unit 28 rotates the support member 26A in an angular range of more than 360 degrees.

The shaft 26B is a shaft extended along a virtual axis F that passes through a position of the support member 26A facing the top portion of the mounting member 16 and that passes through the top portion of the mounting member 16. Therefore, the image capturing units 24 supported by the support member 26A rotate around the head portion H on which the mounting member 16 is mounted. Further, by causing the image capturing units 24 to capture images during the rotation, images of the entire region of the mounting member 16 are captured.

In the present embodiment, when the image capturing units 24 capture images of the mounting member 16, the image capturing is performed in a state in which the markers 22 are set in the arrangement regions P of the holding units 18 on the head portion H and the mounting member 16 is mounted on the head portion H. Therefore, the plurality of image capturing units 24 that rotate around the head portion H along with the rotation of the support member 26A are able to obtain captured images in which the markers 22 and the mounting member 16 set on the head portion H are captured at various angles.

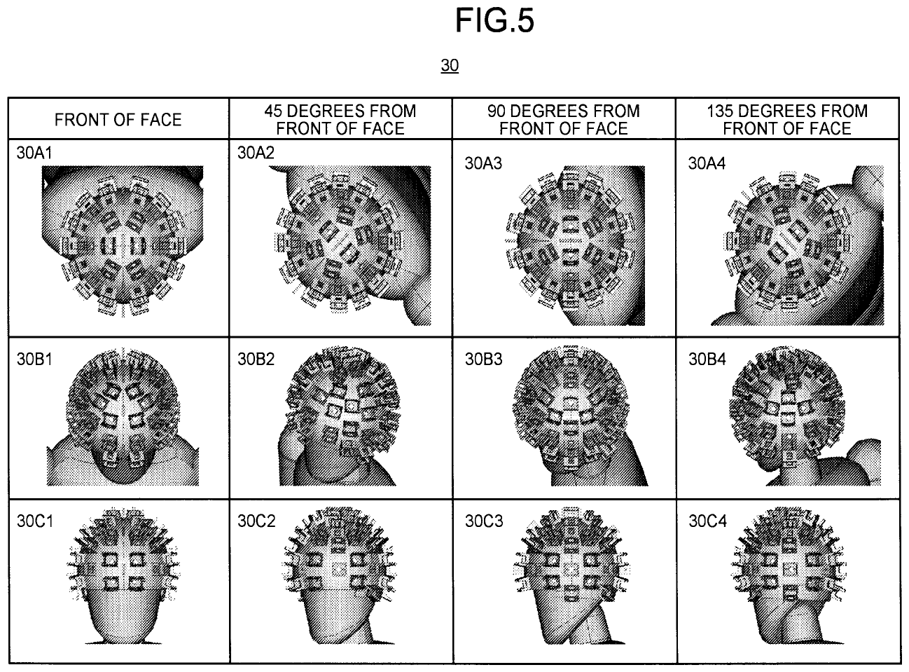

FIG. 5 is a schematic diagram illustrating examples of captured images 30. In FIG. 5, captured images 30A1 to 30A4 are examples of the captured images 30 that are captured by the image capturing unit 24A. Captured images 30B1 to 30B4 are examples of the captured images 30 that are captured by the image capturing unit 24B. Captured images 30C1 to 30C4 are examples of the captured images 30 that are captured by the image capturing unit 24C.

Further, the captured image 30A1, the captured image 30B1, and the captured image 30C1 are examples of the captured images 30 that are obtained when the image capturing units 24 are located in front of a face of the head portion H of the living body B who is sit on the seat portion 14. The captured image 30A2, the captured image 30B2, and the captured image 30C2 are examples of the captured images 30 that are obtained when the image capturing units 24 are located at positions rotated by 45 degrees from the front of the face of the head portion H of the living body B who sits on the seat portion 14. The captured image 30A3, the captured image 30B3, and the captured image 30C3 are examples of the captured images 30 that are obtained when the image capturing units 24 are located at positions rotated by 90 degrees from the front of the face of the head portion H of the living body B who sits on the seat portion 14. The captured image 30A4, the captured image 30B4, and the captured image 30C4 are examples of the captured images 30 that are obtained when the image capturing units 24 are located at positions rotated by 135 degrees from the front of the face of the head portion H of the living body B who sits on the seat portion 14.

Referring back to FIG. 1, explanation is continued. The image capturing units 24 output the captured images 30 to the information processing apparatus 50.

The information processing apparatus 50 controls the moving mechanism unit 25. Further, the information processing apparatus 50 derives the three-dimensional position information on the magnetic sensors 20 held by the holding units 18 with respect to the head portion H by using the captured images 30 acquired from the measurement apparatus 10. Furthermore, the information processing apparatus 50 generates an image that represents a measurement result from the derived three-dimensional position information and the magnetic signal of the head portion H obtained from each of the magnetic sensors 20.

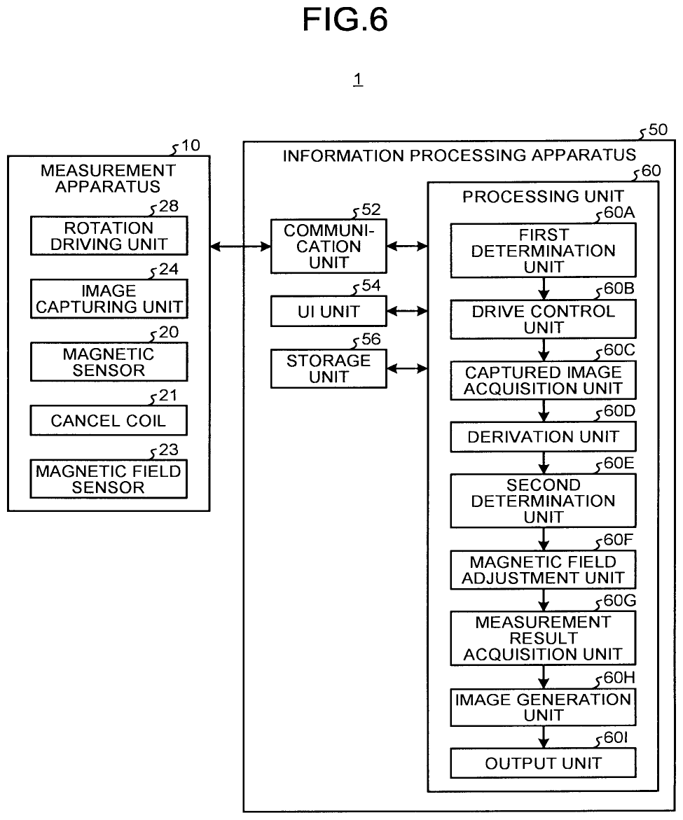

FIG. 6 is an exemplary functional block diagram of the measurement system 1.

The measurement apparatus 10 includes the rotation driving unit 28, the image capturing units 24, the magnetic sensors 20, the cancel coils 21, and a magnetic field sensor 23. The magnetic field sensor 23 is a sensor that measures a magnetic field around the magnetic sensors 20.

The information processing apparatus 50 includes a communication unit 52, a user interface (UI) unit 54, a storage unit 56, and a processing unit 60. The communication unit 52, the UI unit 54, the storage unit 56, and the processing unit 60 are communicably connected to one another.

The communication unit 52 communicates with an external information processing apparatus via a network or the like. In the present embodiment, the communication unit 52 communicates with the measurement apparatus 10.

The UI unit 54 has an input function to receive an operation instruction from a user and a display function to display various kinds of information. The input function is, for example, a keyboard, a pointing device, a microphone, or the like. The display function is, for example, a display, a projection apparatus, or the like. Meanwhile, the UI unit 54 may be a touch panel that has the input function and the display function.

The storage unit 56 stores therein various kinds of information.

The processing unit 60 performs information processing. The processing unit 60 includes a first determination unit 60A, a drive control unit 60B, a captured image acquisition unit 60C, a derivation unit 60D, a second determination unit 60E, a magnetic field adjustment unit 60F, a measurement result acquisition unit 60G, an image generation unit 60H, and an output unit 60I. The first determination unit 60A, the drive control unit 60B, the captured image acquisition unit 60C, the derivation unit 60D, the second determination unit 60E, the magnetic field adjustment unit 60F, the measurement result acquisition unit 60G, the image generation unit 60H, and the output unit 60I are implemented by a single or a plurality of processors, for example. For example, each of the units as described above may be implemented by causing a processor, such as a central processing unit (CPU), to execute a program, in other words, by software. Each of the units as described above may be implemented by a processor, such as a dedicated integrated circuit (IC), in other words, by hardware. Each of the units as described above may be implemented by both of software and hardware. If the plurality of processors are used, each of the processors may implement one of the units or may implement two or more of the units.

Further, at least one of the units as described above may be mounted on a cloud computing server that performs a process on cloud computing.

The first determination unit 60A determines whether the mounting member 16 that does not hold the magnetic sensors 20 is mounted on the head portion H.

For example, a user, such as a person who is responsible for measurement, mounts the mounting member 16 from which all of the magnetic sensors 20 are detached onto the head portion H of the subject who is sitting on the seat portion 14 of the measurement apparatus 10, and inputs information indicating mounting completion. For example, as illustrated in FIG. 1, the mounting member 16 is mounted on the head portion H of the living body B as the subject who is sitting on the seat portion 14. Meanwhile, it is preferable to fix the mounting member 16 to the head portion H with a band member or the like to prevent the mounting member 16 from moving from the head portion H even if the head portion H of the subject slightly moves. Furthermore, if hair of the head portion H of the subject may disturb the measurement, it is preferable to put a cap that fits the head portion H onto the head portion H to reduce a thickness of the hair and thereafter mount the mounting member 16 on the head portion H.

The user operates the UI unit 54 to input the information indicating mounting completion. The first determination unit 60A determines whether the information indicating mounting completion is received from the UI unit 54 to determine whether the mounting member 16 that does not hold the magnetic sensors 20 is mounted the head portion H.

Further, the first determination unit 60A determines whether the markers 22 are arranged in the arrangement regions P of the head portion H. The user arranges the markers 22 in the arrangement regions P of the holding units 18 of the mounting member 16 on the head portion H. It is sufficient for the user to arrange the markers 22 in the arrangement regions P of the holding units 18 that are adopted as targets to which the magnetic sensors 20 are mounted among the plurality of holding units 18 arranged in the mounting member 16. Then, after arranging the markers 22, the user operates the UI unit 54 to input information indicating completion of arrangement of the markers 22. The first determination unit 60A determines whether the information indicating completion of arrangement of the markers 22 is received from the UI unit 54 to determine whether the markers 22 are arranged in the arrangement regions P on the head portion H.

Meanwhile, in some cases, only a partial region of the mounting member 16 may be adopted as a region in which a magnetic signal is to be measured on the head portion H. In this case, it is sufficient to arrange the markers 22 in the arrangement regions P of the holding units 18 that are adopted as targets for holding the magnetic sensors 20 among the plurality of holding units 18 arranged in the mounting member 16.

Furthermore, at this time, the image capturing units 24 may capture images of magnetic markers that are separately arranged on the head portion H, in addition to capturing images of the markers 22 that are used to obtain the three-dimensional position information on the magnetic sensors 20. The magnetic markers may be used for positional alignment when a magnetoencephalograph (MEG) image that is generated based on magnetic signals of the magnetic sensors 20, a separately captured magnetic resonance imaging (MRI) image of the head portion H, and the like are synthesized. In this case, the user may further set the magnetic markers on the head portion H.

The drive control unit 60B controls drive of the rotation driving unit 28. The drive control unit 60B rotates the rotation driving unit 28 such that an angular range in which the support member 26A rotates is equal to or larger than 360 degrees. Through the control by the drive control unit 60B, the support member 26A that supports the plurality of image capturing units 24 rotates in the angular range of more than 360 degrees by using the shaft 26B as the rotation axis. If rotation drive of the support member 26A is started due to the control by the rotation driving unit 28, the image capturing units 24 start to capture images. Meanwhile, the image capturing units 24 may start to capture images due to control by the processing unit 60.

For example, the rotation driving unit 28 stops rotation of the support member 26A every time the support member 26A rotates by a predetermined angle. The image capturing unit 24A and the image capturing unit 24B arranged on the support member 26A simultaneously or sequentially capture images of the head portion H on which the mounting member 16 is mounted, every time the rotation of the support member 26A is stopped. Therefore, by repetition of a series of processes including rotation of the support member 26A by the predetermined angle, stop of the rotation of the support member 26A, and image capturing by the image capturing units 24, images of all of the arranged markers 22 are captured.

Meanwhile, in some cases, the captured images 30 in which some parts of the markers 22 are hidden by the holding units 18 may be obtained when the images of the markers 22 are captured. However, as described above with reference to FIG. 2B, the openings 18D are arranged on the side walls 18C of the holding units 18. Therefore, at least parts of the markers 22 are captured by the image capturing units 24. In other words, it is possible to increase the number of pixels of the markers 22 to be captured as compared to a configuration in which the openings 18D are not arranged.

The captured image acquisition unit 60C acquires the captured images 30 captured by the image capturing units 24. By causing the image capturing units 24 to perform image capturing during rotation of the support member 26A, it is possible to obtain the plurality of captured images 30 as illustrated in FIG. 5, for example.

As illustrated in FIG. 5, in some cases, the captured images 30 in which the markers 22 are hidden by shadows caused by the mounting member 16 or the configurations of the holding units 18 rather than the visual fields of the image capturing units 24 and some parts of the markers 22 are missing may be obtained. However, the image capturing units 24 rotate around the mounting member 16 mounted on the head portion H along with the rotation of the support member 26A that supports the image capturing units 24. Therefore, as illustrated in FIG. 5, along with the rotation, the marker 22 that is lost in an image captured at a rotation position is captured without being lost at another rotation position. The drive control unit 60B adjusts angular intervals of rotation angles at which the image capturing units 24 capture images, so that it is possible to reduce missing areas of the markers 22.

Meanwhile, if a configuration for obtaining the captured images 30 that are captured while the image capturing units 24 rotate around the head portion H once is adopted, it is sufficient to set the rotation angle of the support member 26 to reach 360 degrees in conjunction with a viewing angle of each of the image capturing units 24. Further, if a configuration for obtaining the captured images 30 that are captured while the image capturing units 24 rotate around the head portion H more than once is adopted, it is sufficient to set the rotation angle of the support member 26 to reach 360 degrees or more in conjunction with the viewing angle of each of the image capturing units 24.

Furthermore, if the magnetic sensors 20 are held by only some of the holding units 18 arranged in the mounting member 16, the markers 22 are arranged in only the arrangement regions P of the some of the holding units 18 on the head portion H. In this case, the rotation angle of the support member 26 may be set to reach 360 degrees or less in conjunction with the viewing angle of each of the image capturing units 24.

In other words, it is sufficient for the drive control unit 60B to rotate the support member 26 by a rotation angle that is needed to derive accurate three-dimensional position coordinate information on the markers 22 arranged on the head portion H.

Referring back to FIG. 6, explanation is continued. The derivation unit 60D derives the three-dimensional position information on the magnetic sensors 20 held by the holding units 18 with respect to the head portion H, on the basis of the captured images 30 of the markers 22.

The derivation unit 60D extracts the markers 22 from the captured images 30 that are obtained by capturing images of the mounting member 16 in various angular directions and that include the distance information. Meanwhile, it is assumed that the information processing apparatus 50 measures, in advance, an absolute three-dimensional position coordinate of at least one of the markers 22 by using a three-dimensional digitizer or the like.

Then, the derivation unit 60D derives the three-dimensional position information on each of the extracted markers 22 by using the iterative closest point (ICP) algorithm. The ICP algorithm is a method of obtaining a solution that minimizes an error function through iterative calculation by using a portion that is redundantly measured among a plurality of distance images.

The derivation unit 60D obtains, from the three-dimensional position information on an outline of any of the markers 22, a position of center of gravity of the marker 22 as the three-dimensional position coordinate of the marker 22. Meanwhile, the derivation unit 60D may first derive the three-dimensional position information on the outline from a continuous curve or a continuous straight line that forms the outline of the marker 22, and thereafter obtain the position of the center of gravity of the marker 22 as the three-dimensional position coordinate of the marker 22. Further, the derivation unit 60D may obtain the position of the center of gravity in accordance with the shape of the magnetic sensor 20 from three-dimensional position information on outline of several points of the marker 22, and adopt the position of the center of gravity as the three-dimensional position coordinate of the marker 22. Furthermore, it may be possible to obtain an identifiable point, such as a position of a top point of a side that forms the outline of the marker 22, instead of the position of the center of gravity, as the three-dimensional position coordinate of the marker 22.

Then, the derivation unit 60D obtains the three-dimensional position coordinate of each of the other markers 22 from relative position information on the other markers 22, with reference to the obtained three-dimensional position coordinate of the marker 22.

Positional alignment with a real space may be performed by obtaining a three-dimensional position coordinate in the real space on the basis of a relative three-dimensional position coordinate with respect to the marker 22 that is used as a reference, by using a three-dimensional digitizer.

As described above, the markers 22 have shapes, such as circular shapes or rectangular shapes, that are formed of straight lines or curved lines and that have thicknesses. Therefore, in some cases, all or a part of the markers 22 are not captured due to shadows caused by the configurations of the holding units 18 when the image capturing units 24 capture images. However, in the present embodiment, the holding units 18 include the openings 18D and the captured image acquisition unit 60C acquires the plurality of captured images 30 for each of the markers 22. Therefore, the derivation unit 60D is able to accurately derive the three-dimensional position information on each of the markers 22.

Further, if a magnetic marker is included in any of the captured images 30, a three-dimensional position coordinate of each of the magnetic markers arranged on the head portion H may be derived in the same manner.

The second determination unit 60E determines whether holding of the magnetic sensors 20 by the holding units 18 is completed.

For example, after deriving the three-dimensional position information on each of the markers 22, the derivation unit 60D performs power control of turning off power of each of the rotation driving unit 28 and the image capturing units 24, and the derivation unit 60D displays information indicating completion of derivation of the three-dimensional position on the UI unit 54. If the information indicating completion of derivation of the three-dimensional position is displayed on the UI unit 54, the user removes the markers 22 from the head portion H of the living body B, and sets the magnetic sensors 20 on the mounting member 16 at the positions of the markers 22. In this case, the magnetic sensors 20 are mounted such that a gap between each of the magnetic sensors 20 and the scalp of the head portion H is reduced to nearly zero, in other words, the magnetic sensors 20 come into contact with the scalp of the head portion H.

Meanwhile, when the magnetic sensors 20 are set to the holding units 18, it is preferable to perform setting such that the mounting member 16 is not moved relative to the head portion H. After all of the magnetic sensors 20 that are holding targets are held by the holding units 18, the user operates the UI unit 54 to input information indicating completion of holding of the magnetic sensors 20. Upon receiving the information indicating the completion of the holding, the second determination unit 60E determines that the holding of the magnetic sensors 20 by the holding units 18 is completed.

The magnetic field adjustment unit 60F adjusts a magnetic field. The magnetic field adjustment unit 60F performs feedback control of causing electrical currents to flow through the cancel coils 21 and setting current values of the cancel coils 21 such that a magnetic field measurement result of the magnetic field sensor 23 reaches a desired environmental magnetic field or less.

The measurement result acquisition unit 60G acquires a magnetic signal that is measured under an environment at a predetermined environmental magnetic field or less, from each of the magnetic sensors 20 held by the holding units 18.

The image generation unit 60H generates an image that represents a measurement result by using the three-dimensional position information on each of the magnetic sensors 20 that is derived by the derivation unit 60D and the magnetic signal that is obtained by each of the magnetic sensors 20 and acquired by the measurement result acquisition unit 60G.

For example, the image generation unit 60H generates an MEG image that represents a position of the head portion H at which the magnetic signal is generated, by using the three-dimensional position information on each of the magnetic sensors 20 and the magnetic signal obtained by each of the magnetic sensors 20.

Further, the image generation unit 60H may further generate an image in which the MEG image and an MRI image of the head portion H that is separately captured are synthesized by using, for positional alignment, the three-dimensional position information on the magnetic marker included in the captured image 30 acquired by the captured image acquisition unit 60C.

Meanwhile, in the present embodiment, a case will be described in which the measurement target portion is the head portion H, for example. Therefore, a case will be described in which the image generation unit 60H generates an MEG image, for example. However, it is sufficient for the image generation unit 60H to generate an image corresponding to the measurement target portion of the living body B. For example, the image generation unit 60H may generate a magnetocardiograph (MCG) image, a magnetospinography (MSG) image, or the like in accordance with the measurement target portion.

The output unit 60I outputs the image that represents the measurement result and that is generated by the image generation unit 60H to the UI unit 54. Further, the output unit 60I may output an image that represents the measurement result to an external information processing apparatus via the communication unit 52.

A flow of information processing performed by the information processing apparatus 50 of the present embodiment will be described below.

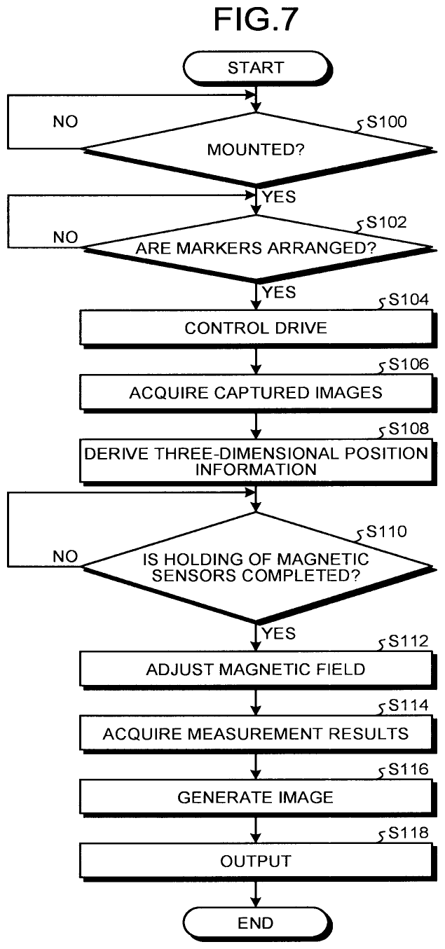

FIG. 7 is a flowchart illustrating an example of the flow of the information processing performed by the information processing apparatus 50 of the present embodiment.

The first determination unit 60A determines whether the mounting member 16 that does not hold the magnetic sensors 20 is mounted on the head portion H (Step S100). The first determination unit 60A repeats negative determination until it is determined that the information indicating mounting completion is received from the UI unit 54 (NO at Step S100). If the first determination unit 60A determines that the information indicating mounting completion is received from the UI unit 54 (YES at Step S100), the process goes to Step S102.

At Step S102, the first determination unit 60A determines whether the markers 22 are arranged in the arrangement regions P (Step S102). The first determination unit 60A repeats negative determination until it is determined that the information indicating completion of arrangement of the markers 22 is received from the UI unit 54 (NO at Step S102). If it is determined that the information indicating completion of arrangement of the markers 22 is received (YES at Step S102), the process goes to Step S104.

At Step S104, the drive control unit 60B controls drive of the rotation driving unit 28 (Step S104). The drive control unit 60B causes the rotation driving unit 28 to stop rotation of the support member 26A every time the support member 26A rotates by the predetermined angle. Then, the drive control unit 60B causes an image of the head portion H, on which the markers 22 are arranged and the mounting member 16 is mounted, to be captured every time the rotation of the support member 26A is stopped. Therefore, by repetition of a series of processes including rotation of the support member 26A by the predetermined angle, stop of the rotation of the support member 26A, and image capturing by the image capturing units 24, images of all of the arranged markers 22 are captured.

The captured image acquisition unit 60C acquires the captured images 30 that are captured by the image capturing units 24 through the drive control performed at Step S104 (Step S106). For example, the captured image acquisition unit 60C acquires the plurality of captured images 30 as illustrated in FIG. 5.

The derivation unit 60D derives the three-dimensional position information on the magnetic sensors 20 held by the holding units 18 with respect to the head portion H, on the basis of the captured images 30 of the markers 22 acquired at Step S106 (Step S108).

The second determination unit 60E determines whether holding of the magnetic sensors 20 by the holding units 18 is completed (Step S110). The second determination unit 60E repeats negative determination until it is determined that the information indicating completion of the holding is received from the UI unit 54 (NO at Step S110). If it is determined that the information indicating the completion of the holding completion is received (YES at Step S110), the process goes to Step S112.

At Step S112, the magnetic field adjustment unit 60F adjusts the magnetic field (Step S112). The magnetic field adjustment unit 60F performs feedback control of causing electrical currents to flow through the cancel coils 21 and setting current values of the cancel coils 21 such that a magnetic field measurement result of the magnetic field sensor 23 reaches a desired environmental magnetic field or less.

The measurement result acquisition unit 60G acquires the measurement results from the magnetic sensors 20 (Step S114). The measurement result acquisition unit 60G acquires, as the measurement result, the magnetic signal measured by each of the magnetic sensors 20 under an environment at the predetermined environmental magnetic field through the process at Step S112, form each of the magnetic sensors 20 held by the holding units 18 (Step S114).

The image generation unit 60H generates an image that represents the measurement result by using the three-dimensional position information on each of the magnetic sensors 20 that is derived at Step S108 and the magnetic signal that is obtained by each of the magnetic sensors 20 and that is acquired at Step S114 (Step S116). For example, the image generation unit 60H generates an MEG image that represents a position of the head portion H at which the magnetic signal is generated, by using the three-dimensional position information on each of the magnetic sensors 20 and the magnetic signal obtained by each of the magnetic sensors 20. Further, the image generation unit 60H may further generate an image in which the MEG image and an MRI image of the head portion H that is separately captured are synthesized.

The output unit 60I outputs the image that represents the measurement result and that is generated at Step S116 to at least one of the UI unit 54 and an external information processing apparatus (Step S118). Then, the routine is finished.

As described above, the measurement system 1 of the present embodiment includes the mounting member 16, the image capturing units 24, the moving mechanism unit 25, and the derivation unit 60D. The mounting member 16 is mounted on the head portion H that is one example of the measurement target portion of the living body B, and includes the plurality of holding units 18 for holding the plurality of magnetic sensors 20 such that the magnetic sensors 20 face the head portion H. The image capturing units 24 capture images of the markers 22 that are arranged in the arrangement regions P of the holding units 18 of the mounting member 16 mounted on the head portion H, on the head portion H. The moving mechanism unit 25 moves the image capturing units 24 relative to the head portion H on which the mounting member 16 is mounted. The derivation unit 60D derives the three-dimensional position information on the magnetic sensors 20 held by the holding units 18 with respect to the head portion H, on the basis of the captured images 30 of the markers 22.

In this manner, in the measurement system 1 of the present embodiment, the moving mechanism unit 25 moves the image capturing units 24 relative to the head portion H on which the mounting member 16 is mounted. The image capturing units 24 capture images of the markers 22 arranged in the arrangement regions P of the holding units 18 of the mounting member 16 that is mounted on the head portion H.

Therefore, in the measurement system 1 of the present embodiment, it is possible to automatically capture images of the markers 22, which are arranged in the arrangement regions P of the holding units 18 on the head portion H, in various directions. Further, the derivation unit 60D derives the three-dimensional position information on the magnetic sensors 20 held by the holding units 18 with respect to the head portion H, on the basis of the captured images 30 of the markers 22. Therefore, in the measurement system 1 of the present embodiment, even if at least a part of the markers 22 in the single captured image 30 is lost, it is possible to accurately derive the three-dimensional position information on the markers 22 by using the captured images 30 that are captured in various directions.

Consequently, in the measurement system 1 of the present embodiment, it is possible to obtain the three-dimensional position information with high accuracy as the measurement position of the magnetic signal in the measurement target portion of the living body B.

Furthermore, in the measurement system 1 of the present embodiment, an image, such as an MEG image, that represents the measurement result is generated by using the three-dimensional position information on each of the magnetic sensors 20 that is accurately derived and the magnetic signal that is obtained by each of the magnetic sensors 20. Therefore, in the measurement system 1 of the present embodiment, it is possible to provide a highly-accurate image that represents a position at which the magnetic signal is generated in the measurement target portion, such as the head portion H.

Moreover, the holding units 18 of the present embodiment have shapes such that at least parts of the markers 22 arranged in the arrangement regions P on the head portion H are not located in blind areas of the image capturing units 24. Therefore, the derivation unit 60D is able to derive the three-dimensional position information on the markers 22 with higher accuracy, on the basis of the captured images 30 of the markers 22.

Meanwhile, in the present embodiment, the case has been described, as one example, in which the measurement apparatus 10 includes the seat portion 14 and the subject as the living body B is subjected to measurement of the magnetic signal while the subject is sitting on the seat portion 14. However, the measurement apparatus 10 may measure the magnetic signal of the subject in a standing position or in a supine position. In the case of the standing position, the same configuration as illustrated in FIG. 1 is applicable. In the case of the supine position, it is sufficient to provide a board, such as a bed, on which the subject can lie down instead of the seat portion 14. Furthermore, in this case, a configuration in which the mounting member 16 is fixed to the housing 12, the head portion H of the subject is inserted in the mounting member 16 with movement of the subject, and the mounting member 16 is mounted on the head portion H may be applicable.

### Second Embodiment

In a second embodiment, a configuration in which a moving mechanism unit 25B different from the first embodiment is provided as the moving mechanism unit 25 will be described. Meanwhile, in the present embodiment, the same functional components as those of the first embodiment as described above are denoted by the same reference symbols and detailed explanation thereof will be omitted.

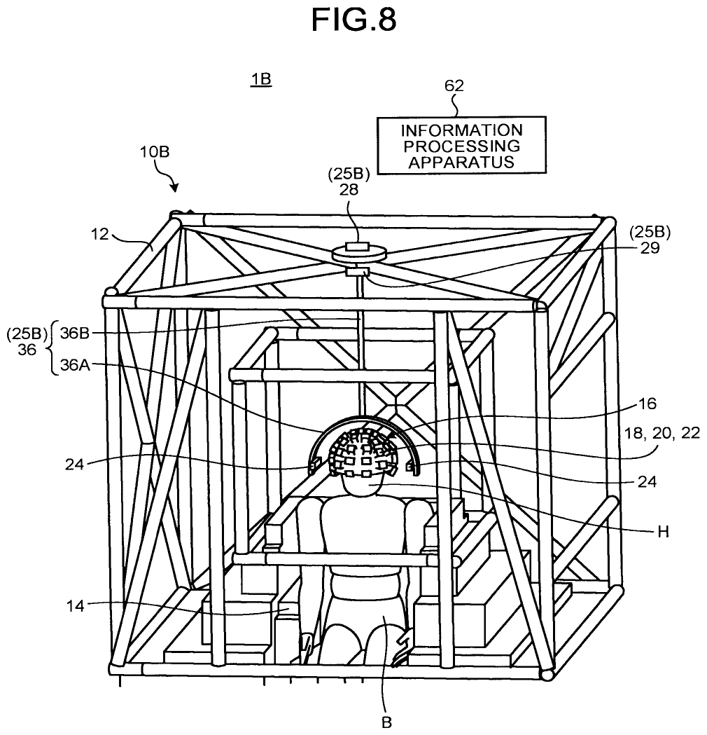

FIG. 8 is a schematic diagram of a measurement system 1B according to the present embodiment. The measurement system 1B includes a measurement apparatus 10B and an information processing apparatus 62. The measurement apparatus 10B and the information processing apparatus 62 are communicably connected to each other.

The measurement apparatus 10B has the same configuration as the measurement apparatus 10 of the first embodiment except that the measurement apparatus 10B includes the moving mechanism unit 25B instead of the moving mechanism unit 25. Therefore, differences from the first embodiment as described above will be described in detail below.

The moving mechanism unit 25B is, similarly to the moving mechanism unit 25, a moving mechanism that moves the image capturing units 24 relative to the head portion H of the living body B on which the mounting member 16 is mounted. In the present embodiment, the moving mechanism unit 25B rotates and moves the image capturing units 24 in a direction along a central axis that is a rotation axis of the rotation.

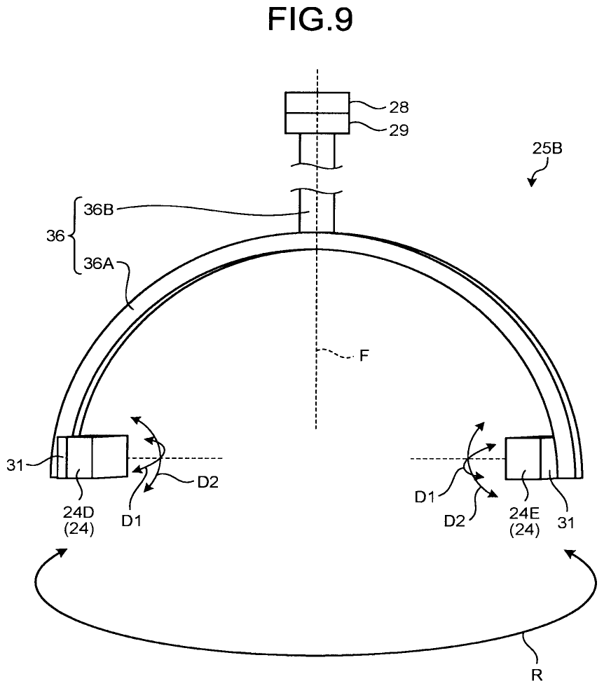

FIG. 9 is an enlarged schematic diagram of the moving mechanism unit 25B. The moving mechanism unit 25B includes a support member 36, the rotation driving unit 28, and a movement driving unit 29. The support member 36 includes a support member 36A and a shaft 36B.

The support member 36A is a member that supports the image capturing units 24 such that the image capturing units 24 face the head portion H. In other words, the support member 36A supports the image capturing units 24 such that the image capturing units 24 are able to capture images of the markers 22 on the head portion H.

In the present embodiment, the support member 36A has a shape that is extended from the top portion of the mounting member 16 along the outer shape of the mounting member 16. As described above, the mounting member 16 has a semispherical shape. Therefore, in the present embodiment, the support member 36A has a circular arc shape that conforms to the semispherical outer shape of the mounting member 16. It is preferable that a curvature of the support member 36A approximately matches a curvature of the outer shape of the mounting member 16.

In the present embodiment, the support member 36A has a shape that is extended from the top portion of the mounting member 16 and is branched in two opposing directions toward an opening, in which the head portion H is inserted, along the outer shape of the mounting member 16. Further, the support member 36A supports the image capturing units 24 at opposing positions across the mounting member 16.

In the present embodiment, the support member 36A holds an image capturing unit 24D in one end portion in an extending direction of the support member 36A having a circular arc shape, and holds an image capturing unit 24E at the other end portion in the extending direction. The image capturing unit 24D and the image capturing unit 24E are one example of the image capturing units 24.

Each of the image capturing unit 24D and the image capturing unit 24E is held so as to be able to change an image capturing direction at each of the positions held by the support member 36A. Specifically, as illustrated in FIG. 9, each of the image capturing unit 24D and the image capturing unit 24E is held so as to be able to change the image capturing direction to a direction D1 along a rotation axis F and a direction D2 that intersects with the direction D1. Specifically, a direction driving unit 31 is arranged in each of the image capturing unit 24D and the image capturing unit 24E. The direction driving unit 31 changes the image capturing direction of each of the image capturing unit 24D and the image capturing unit 24E along the direction D1 and the direction D2.

The shaft 36B is a linear bar-shaped member. One end portion of the shaft 36B is connected to the support member 36A, and the other end portion is connected to the housing 12 via the rotation driving unit 28 and the movement driving unit 29.

The rotation driving unit 28 rotates the support member 36A about the head portion H as a rotation center. Specifically, in the present embodiment, the rotation driving unit 28 rotates the support member 36A about the shaft 36B as a rotation axis. Therefore, the shaft 36B and the support member 36A are driven to rotate in the direction of arrow R. The rotation driving unit 28 rotates the support member 36A in an angular range of more than 360 degrees.

The shaft 36B is a shaft extended along the virtual axis F that passes through a position of the support member 36A facing the top portion of the mounting member 16 and that passes through the top portion of the mounting member 16. Therefore, the image capturing units 24 supported by the support member 36A rotate around the head portion H on which the mounting member 16 is mounted. Further, by causing the image capturing units 24 to capture images during the rotation, images of the entire region of the mounting member 16 are captured.

Furthermore, in the present embodiment, the movement driving unit 29 moves the support member 36A in a direction along the rotation axis of the rotation of the support member 36A. Specifically, the movement driving unit 29 moves the support member 36A in an extending direction of the shaft 36B that serves as the rotation axis of the support member 36A. In other words, in the example illustrated in FIG. 9, the movement driving unit 29 moves the support member 36A along a direction of arrow X.

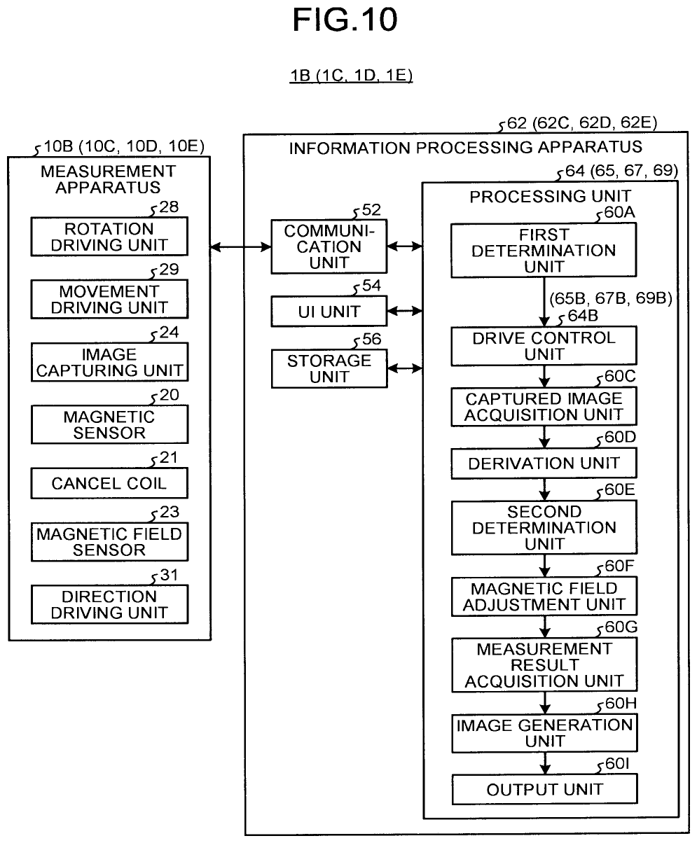

FIG. 10 is an exemplary functional block diagram of the measurement system 1B.

The measurement apparatus 10B includes the rotation driving unit 28, the image capturing units 24, the magnetic sensors 20, the cancel coils 21, the magnetic field sensor 23, the movement driving unit 29, and the direction driving unit 31.

The information processing apparatus 62 includes the communication unit 52, the UI unit 54, the storage unit 56, and a processing unit 64. The communication unit 52, the UI unit 54, the storage unit 56, and the processing unit 64 are communicably connected to one another. The information processing apparatus 62 is the same as the processing unit 60 of the first embodiment as described above except that the information processing apparatus 62 includes the processing unit 64 instead of the processing unit 60.

The processing unit 64 includes the first determination unit 60A, a drive control unit 64B, the captured image acquisition unit 60C, the derivation unit 60D, the second determination unit 60E, the magnetic field adjustment unit 60F, the measurement result acquisition unit 60G, the image generation unit 60H, and the output unit 60I. The processing unit 64 is the same as the processing unit 60 of the first embodiment as described above except that the processing unit 64 includes the drive control unit 64B instead of the drive control unit 60B.

The drive control unit 64B controls drive of the rotation driving unit 28, the movement driving unit 29, and the direction driving unit 31. The drive control unit 64B rotates the rotation driving unit 28 such that an angular range in which the support member 36A rotates is equal to or larger than 180 degrees. Further, the drive control unit 64B controls the movement driving unit 29 such that the support member 36A moves along the extending direction of the shaft 36B. Furthermore, the drive control unit 64B controls the direction driving unit 31 such that the image capturing directions of the image capturing units 24 are changed.

Therefore, the image capturing unit 24D and the image capturing unit 24E that are the two image capturing units 24 arranged at both end portions in the extending direction of the support member 36A rotate about the shaft 26B as the rotation axis, move along an extending direction of the shaft 26B, and capture images of the markers 22 on the mounting member 16 while changing the image capturing directions.

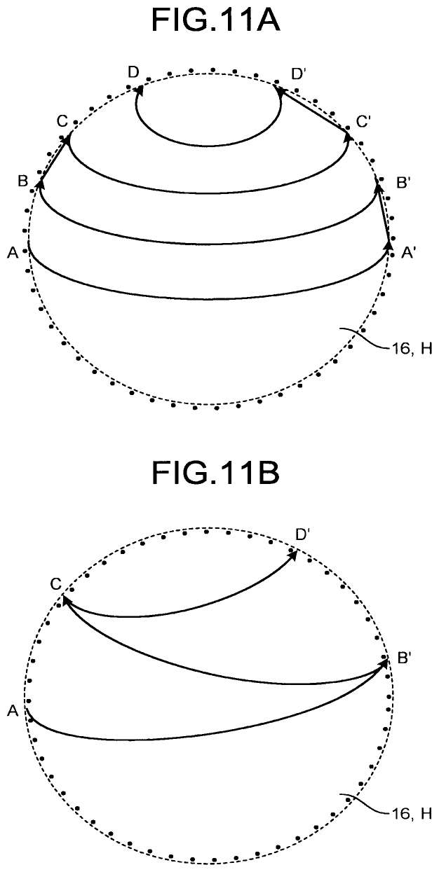

FIG. 11A is a diagram for explaining an example of a movement pattern of one of the image capturing units 24 under the control of the drive control unit 64B. FIG. 11A illustrates an example of the movement pattern of the image capturing unit 24D.

As illustrated in FIG. 11A, for example, the drive control unit 64B controls the rotation driving unit 28 and the movement driving unit 29 such that the image capturing unit 24D moves to a position A, a position A′, a position B′, a position B, a position C, a position C′, a position D′, and a position D in this order.

Specifically, the drive control unit 64B controls the rotation driving unit 28 such that the position of the image capturing unit 24D is moved from the position A to the position A′ by 180 degrees in a clockwise direction. Then, the drive control unit 64B controls the movement driving unit 29 such that the image capturing unit 24D is moved from the position A′ to the position B′ in an extending direction of the rotation axis of the shaft 36B. Subsequently, the drive control unit 64B controls the rotation driving unit 28 such that the image capturing unit 24D is rotated from the position B′ to the position B by 180 degrees in a counterclockwise direction. Furthermore, the drive control unit 64B controls the movement driving unit 29 such that the image capturing unit 24D is moved from the position B to the position C in the extending direction of the rotation axis of the shaft 36B. Then, the drive control unit 64B controls the rotation driving unit 28 such that the image capturing unit 24D is rotated from the position C to the position C′ by 180 degrees in the clockwise direction. Moreover, the drive control unit 64B controls the movement driving unit 29 such that the image capturing unit 24D moves from the position C′ to the position D′ in the extending direction of the rotation axis of the shaft 36B. Then, the drive control unit 64B controls the rotation driving unit 28 such that the image capturing unit 24D is rotated from the position D′ to the position D by 180 degrees in the counterclockwise direction.

By the rotation drive and the movement drive of the support member 36A, the image capturing unit 24E that is arranged in the other end portion of the support member 36A follows the same movement pattern on a back surface side of the mounting member 16.

Further, the drive control unit 64B causes the image capturing units 24 to acquire the captured images 30 by performing image capturing every time the support member 36A rotates by the predetermined angle and moves in the extending direction of the shaft 36B.

Furthermore, the drive control unit 64B causes the image capturing units 24 to acquire the captured images 30 in each of the image capturing directions while changing the image capturing direction of the image capturing unit 24D every time the support member 36A moves in the extending direction of the shaft 36B.

Therefore, the image capturing units 24 capture images of all of the markers 22 arranged on the head portion H.

FIG. 11B is a diagram for explaining another example of the movement pattern of one of the image capturing units 24 under the control of the drive control unit 64B. FIG. 11B illustrates an example of the movement pattern of the image capturing unit 24D.

As illustrated in FIG. 11B, for example, the drive control unit 64B controls the rotation driving unit 28 and the movement driving unit 29 such that the image capturing unit 24D moves to the position A, the position B′, the position B, the position C, and the position D′ in this order. In other words, the drive control unit 64B moves the support member 36A along the extending direction of the shaft 36B while rotating the support member 36A.

By the rotation drive and the movement drive of the support member 36A, the image capturing unit 24E that is arranged in the other end portion of the support member 36A follows the same movement pattern on the back surface side of the mounting member 16.

Further, the drive control unit 64B also controls the direction driving unit 31 while rotating and moving the support member 36A.

Then, the drive control unit 64B causes the support member 36A to rotate and move, and causes the image capturing units 24 to capture the captured images 30 at different positions and in different image capturing directions while causing the direction driving unit 31 to change the image capturing directions of the image capturing units 24. Meanwhile, at the time of image capturing by the image capturing units 24, the rotation drive and the movement drive of the support member 36A and the drive of the direction driving unit 31 are performed in a standstill state.

Therefore, the image capturing units 24 capture images of all of the markers 22 arranged on the head portion H.

FIG. 11C is a diagram for explaining another example of the movement pattern of one of the image capturing units 24 under the control of the drive control unit 64B. FIG. 11C illustrates an example of the movement pattern of the image capturing unit 24D.

As illustrated in FIG. 11C, for example, the drive control unit 64B controls the rotation driving unit 28 and the movement driving unit 29 such that the image capturing unit 24D repeats a series of movement patterns such that the image capturing unit 24D first moves from the position A to the position D along the extending direction of the shaft 36B, and thereafter the image capturing unit 24D is rotated by the predetermined angle and moves in an opposite direction of the extending direction of the shaft 36B.

By the rotation drive and the movement drive of the support member 36A, the image capturing unit 24E that is arranged in the other end portion of the support member 36A follows the same movement pattern on the back surface side of the mounting member 16.

Further, the drive control unit 64B also controls the direction driving unit 31 while rotating and moving the support member 36A.

Then, the drive control unit 64B causes the image capturing units 24 to capture the captured images 30 at different positions and in different image capturing directions while rotating and moving the support member 36A and causing the direction driving unit 31 to change the image capturing directions of the image capturing units 24.

Therefore, the image capturing units 24 capture images of all of the markers 22 arranged on the head portion H.

Meanwhile, the movement pattern of the shaft 36B, in other words, the image capturing units 24, by the drive control unit 64B is not limited to the examples illustrated in FIG. 11A to FIG. 11C. It is sufficient for the drive control unit 64B to select a movement pattern that is appropriate for measurement of the head portion H, and use the selected movement pattern to control the rotation driving unit 28, the movement driving unit 29, and the direction driving unit 31.

If a configuration for obtaining the captured images that are captured while the image capturing units 24 rotate around the head portion H once is adopted, it is sufficient to set a rotation angle of the support member 36 to reach 180 degrees in conjunction with the viewing angle of each of the image capturing units 24. Further, if a configuration for obtaining the captured images 30 that are captured while the image capturing units 24 rotate around the head portion H more than once is adopted, it is sufficient to set the rotation angle of the support member 36 to reach 180 degrees or more in conjunction with the viewing angle of each of the image capturing units 24. It is sufficient for the drive control unit 64B to rotate the support member 36 by a rotation angle that is needed to accurately obtain the three-dimensional position coordinates of the markers 22.

A flow of information processing performed by the information processing apparatus 62 of the present embodiment will be described below.

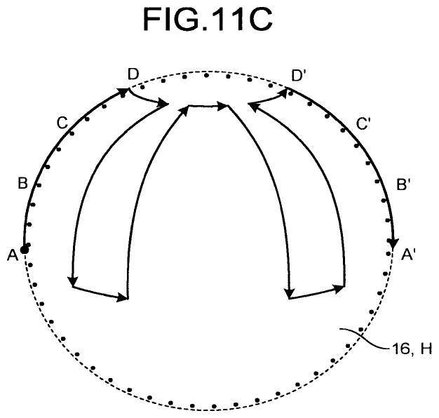

FIG. 12 is a flowchart illustrating an example of the of the information processing performed by the information processing apparatus 62 of the present embodiment.

The information processing apparatus 62 performs processes at Step S200 and Step S202 in the same manner as the processes performed by the information processing apparatus 50 at Step S100 and Step S102.

Subsequently, the drive control unit 64B performs drive control (Step S204). At Step S204, the drive control unit 64B controls the rotation driving unit 28, the movement driving unit 29, and the direction driving unit 31 such that the support member 36A rotates about the shaft 36B as the rotation axis, the support member 36A moves in the extending direction of the shaft 26B, and the image capturing directions are changed. Therefore, the image capturing units 24 acquire the captured images 30 every time the support member 36A rotates by the predetermined angle, the support member 36A moves in the extending direction of the shaft 36B, and the image capturing directions are changed.

Then, the information processing apparatus 62 performs processes from Step S206 to Step S218 in the same manner as the processes performed by the information processing apparatus 50 of the first embodiment at Step S106 to Step S118, and the routine is finished.

As described above, in the measurement system 1B of the present embodiment, the drive control unit 64B causes the support member 36A that supports the image capturing units 24 to rotate about the shaft 36B as the rotation axis, and moves the support member 36A in the extending direction of the shaft 36B. Therefore, in the present embodiment, it is possible to acquire the captured images 30 of all of the markers 22 arranged on the head portion H.

Therefore, the measurement system 1B of the present embodiment is able to acquire the three-dimensional position information with high accuracy, as the measurement position of the magnetic signal in the measurement target portion of the living body B, in the same manner as the embodiment as described above.

### Third Embodiment

In the second embodiment as described above, the case in which the support member 36 of the moving mechanism unit 25B is supported by the housing 12 has been described. In a third embodiment, a case in which the support member 36 is supported by the mounting member 16 will be described.

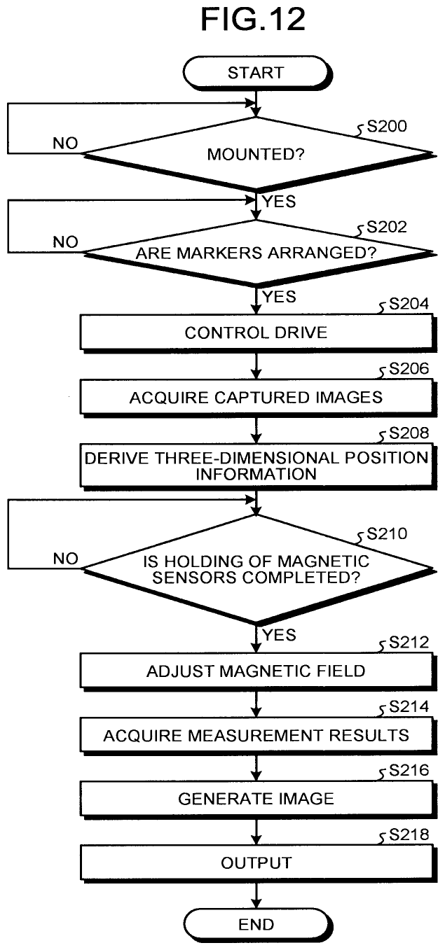

FIG. 13 is a schematic diagram illustrating an example of a moving mechanism unit 25C. The moving mechanism unit 25C is mounted on a measurement apparatus 10C of a measurement system 1C of the present embodiment. Meanwhile, the measurement apparatus 10C of the present embodiment is the same as the measurement apparatus 10 and the measurement apparatus 10B of the embodiments as described above except that the measurement apparatus 10C includes the moving mechanism unit 25C instead of the moving mechanism unit 25 and the moving mechanism unit 25B of the embodiments as described above. Therefore, detailed explanation of mechanisms other than the moving mechanism unit 25C will be omitted.

The moving mechanism unit 25C is a moving mechanism that moves the image capturing units 24 relative to the head portion H of the living body B on which the mounting member 16 is mounted, similarly to the moving mechanism unit 25B. In the present embodiment, the moving mechanism unit 25C rotates the image capturing units 24.

The moving mechanism unit 25C includes a support member 37, the rotation driving unit 28, and the direction driving unit 31. The support member 37 includes a support member 37A and a shaft 37B.

The support member 37A is a member that supports the image capturing units 24 such that the image capturing units 24 face the head portion H. In other words, the support member 37A supports the image capturing units 24 such that the image capturing units 24 are able to capture images of the markers 22 on the head portion H.

In the present embodiment, the support member 37A has a shape that is extended from the top portion of the mounting member 16 along the outer shape of the mounting member 16. As described above, the mounting member 16 has a semispherical shape. Therefore, the support member 37A has a circular arc shape that conforms to the semispherical outer shape of the mounting member 16. It is preferable that a curvature of the support member 37A approximately matches the curvature of the outer shape of the mounting member 16.

The support member 37A has a shape that is extended from the top portion of the mounting member 16 and is branched in two opposing directions toward an opening, in which the head portion H is inserted, along the outer shape of the mounting member 16. Further, the support member 37A supports the plurality of image capturing units 24 in an extending direction of the support member 37A. In the present embodiment, the support member 37A supports image capturing units 24F to 24K as the image capturing units 24. The image capturing unit 24F and the image capturing unit 24K are arranged at one end portion and the other end portion in the extending direction of the support member 37A. The image capturing unit 24H and the image capturing unit 24I are arranged on end portions of the support member 37A on the shaft 37B side. The image capturing unit 24G is arranged between the image capturing unit 24F and the image capturing unit 24H on the support member 37A. Further, the image capturing unit 24J is arranged between the image capturing unit 24I and the image capturing unit 24K on the support member 37A.

The shaft 37B is a linear bar-shaped member. One end portion of the shaft 37B is connected to the support member 37A, and the other end portion is connected to the mounting member 16. Therefore, in the present embodiment, the support member 37 is supported by the mounting member 16.

The rotation driving unit 28 rotates the support member 37A about the head portion H as a rotation center. Specifically, in the present embodiment, the rotation driving unit 28 rotates the support member 37A about the shaft 37B as a rotation axis. Therefore, the shaft 37B and the support member 37A are driven to rotate in the direction of arrow R. The rotation driving unit 28 rotates the support member 37A in an angular range of more than 360 degrees.

The shaft 37B is a shaft that passes through a position of the support member 37A facing the top portion of the mounting member 16 and that passes through the top portion of the mounting member 16. Therefore, the image capturing units 24 supported by the support member 37A rotate around the head portion H on which the mounting member 16 is mounted. Further, by causing the image capturing units 24 to capture images during the rotation, images of the entire region of the mounting member 16 are captured.

For example, the rotation driving unit 28 stops rotation of the support member 37A every time the support member 37A rotates by a predetermined angle. The plurality of image capturing units 24F to 24K arranged on the support member 37A simultaneously or sequentially capture images of the head portion H on which the mounting member 16 is mounted, every time the rotation of the support member 37A is stopped. Therefore, by repetition of a series of processes including rotation of the support member 37A by the predetermined angle, stop of the rotation of the support member 37A, and image capturing by the image capturing units 24, images of all of the arranged markers 22 are captured.

Meanwhile, a weight may be applied to the head portion H of the subject when the configuration in which the mounting member 16 holds the support member 37 is adopted. In this case, it may be possible to adopt a configuration in which the support member 37 is separately supported by a frame to prevent a weight from being applied to the head portion H.

FIG. 10 is a functional block diagram of the measurement system 1C of the present embodiment.

The measurement system 1C includes the measurement apparatus 10C and an information processing apparatus 62C. The measurement apparatus 10C and the information processing apparatus 62C are communicably connected to each other. The functional configuration of the measurement apparatus 10C is the same as that of the measurement apparatus 10B except that the measurement apparatus 10C does not include the movement driving unit 29. The information processing apparatus 62C is the same as the information processing apparatus 62 except that the information processing apparatus 62C includes a processing unit 65 instead of the processing unit 64. The processing unit 65 is the same as the processing unit 64 except that the processing unit 65 includes a drive control unit 65B instead of the drive control unit 64B.

Explanation will be given below with reference to FIG. 13. The drive control unit 65B controls drive of the rotation driving unit 28, similarly to the drive control unit 60B of the first embodiment. The drive control unit 65B rotates the rotation driving unit 28 such that an angular range in which the support member 37A rotates is equal to or larger than 360 degrees. Through the control by the drive control unit 65B, the support member 37A that supports the plurality of image capturing unit 24 rotates in the angular range of more than 360 degrees by using the shaft 37B as the rotation axis. If rotation drive of the support member 37A is started due to the control by the rotation driving unit 28, the image capturing units 24 start to capture images.

For example, the rotation driving unit 28 stops rotation of the support member 37A every time the support member 37A rotates by a predetermined angle. The plurality of image capturing units 24F to 24K arranged on the support member 37A simultaneously or sequentially capture images of the head portion H on which the mounting member 16 is mounted, every time the rotation of the support member 37A is stopped. Therefore, by repetition of a series of processes including rotation of the support member 37A by the predetermined angle, stop of the rotation of the support member 37A, and image capturing by the image capturing units 24, images of all of the arranged markers 22 are captured.

Further, the drive control unit 65B may further cause the direction driving unit 31 to change the image capturing directions of the image capturing units 24. Therefore, the drive control unit 65B stops the rotation of the support member 37A every time the support member 37A rotates by the predetermined angle. Then, the drive control unit 65B controls the direction driving unit 31 every time the rotation of the support member 37A is stopped. Through the control as described above, it is sufficient for the drive control unit 65B to control the image capturing units 24 and the direction driving unit 31 so as to obtain the captured images 30 in a plurality of different image capturing directions that are achieved by rotation as indicated by D1 and D2 in the figure.

Therefore, it is possible to acquire the captured images 30 of all of the markers 22 arranged on the head portion H.

Consequently, the measurement system 1C of the present embodiment is able to obtain the three-dimensional position information with high accuracy, as the measurement position of the magnetic signal in the measurement target portion of the living body B, similarly to the embodiments as described above.

### Fourth Embodiment

In the second embodiment, the case has been described in which the support member 36A is supported by the shaft 36B at a position corresponding to the rotation axis. However, the support member 36A may be supported by shafts at both end portions in the extending direction of the support member 36A.

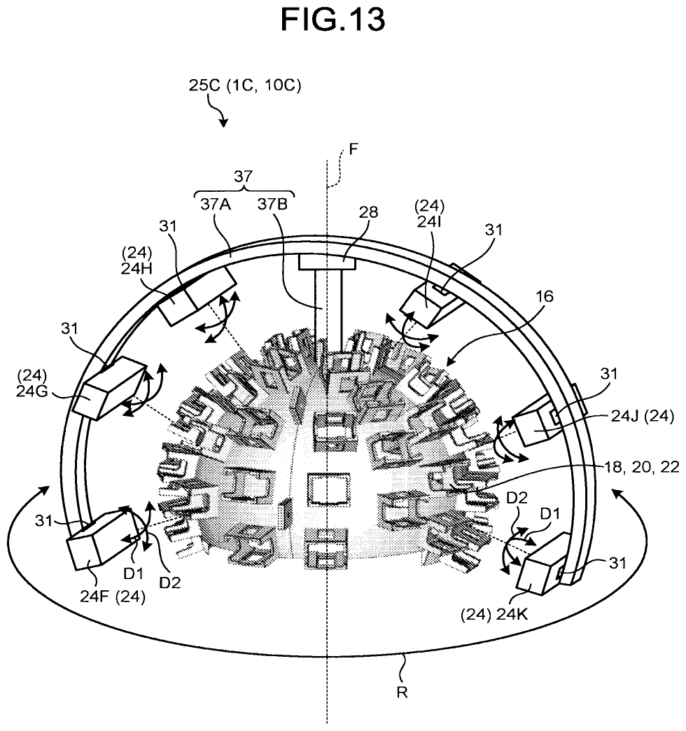

FIG. 14 is a schematic diagram illustrating an example of a moving mechanism unit 25D of the present embodiment. The moving mechanism unit 25D is mounted on a measurement apparatus 10D of a measurement system 1D of the present embodiment. Meanwhile, the measurement apparatus 10D of the present embodiment has the same configuration as those of the measurement apparatus 10, the measurement apparatus 10B, and the measurement apparatus 10C of the embodiments as described above except that the measurement apparatus 10D includes the moving mechanism unit 25D instead of the moving mechanism unit 25, the moving mechanism unit 25B, and the moving mechanism unit 25C of the embodiments as described above. Therefore, detailed explanation of mechanisms other than the moving mechanism unit 25D will be omitted.

The moving mechanism unit 25D is a moving mechanism that moves the image capturing units 24 relative to the head portion H of the living body B on which the mounting member 16 is mounted. In the present embodiment, the moving mechanism unit 25D rotates the image capturing units 24.

The moving mechanism unit 25D includes a support member 38, the rotation driving unit 28, and the direction driving unit 31. The support member 38 includes a support member 38A and a shaft 38B.

The support member 38A is a member that supports the image capturing units 24 such that the image capturing units 24 face the head portion H. In other words, the support member 36A supports the image capturing units 24 such that the image capturing units 24 are able to capture images of the markers 22 on the head portion H.

In the present embodiment, the support member 38A has a shape that is extended from the top portion of the mounting member 16 along the outer shape of the mounting member 16. As described above, the mounting member 16 has a semispherical shape. Therefore, the support member 38A has a circular arc shape that conforms to the semispherical outer shape of the mounting member 16. It is preferable that a curvature of the support member 38A approximately matches the curvature of the outer shape of the mounting member 16.

The support member 38A has a shape that is extended from the top portion of the mounting member 16 and is branched in two opposing directions toward an opening, in which the head portion H is inserted, along the outer shape of the mounting member 16. Further, the support member 38A supports the plurality of image capturing units 24 in an extending direction of the support member 38A. In the present embodiment, the support member 38A supports the plurality of image capturing units 24 at predetermined intervals from one end portion to the other end portion in the extending direction of the support member 38A. FIG. 14 illustrates a mode in which the support member 38A supports image capturing units 24L to 24Q.

The shaft 38B is a linear bar-shaped member. One end portion of the shaft 38B is connected to one end of the support member 38A in the extending direction, and the other end portion is connected to the housing 12 via the rotation driving unit 28.

The rotation driving unit 28 rotates the support member 38A about the shaft 38B as the rotation axis in an angular range of more than 180 degrees. Specifically, if the rotation driving unit 28 rotates, rotation motion is transmitted to the shaft 38B, so that the support member 38A rotates about the shaft 38B as the rotation axis in an angular range of more than 180 degrees. Therefore, the image capturing units 24 supported by the support member 38A move from the face side of the head portion H on which the mounting member 16 is mounted to the back side of the head.

FIG. 10 is a functional block diagram of the measurement system 1D of the present embodiment.

The measurement system 1D includes the measurement apparatus 10D and an information processing apparatus 62D. The measurement apparatus 10D and the information processing apparatus 62D are communicably connected to each other. The functional configuration of the measurement apparatus 10D is the same as that of the measurement apparatus 10B except that the measurement apparatus 10D does not include the movement driving unit 29. The information processing apparatus 62D is the same as the information processing apparatus 62 except that the information processing apparatus 62D includes a processing unit 67 instead of the processing unit 64. The processing unit 67 is the same as the processing unit 64 except that the processing unit 67 includes a drive control unit 67B instead of the drive control unit 64B.

Explanation will be given below with reference to FIG. 14. The drive control unit 67B controls drive of the rotation driving unit 28, similarly to the drive control unit 60B of the first embodiment. The drive control unit 67B rotates the rotation driving unit 28 such that an angular range in which the support member 38A rotates is equal to or larger than 180 degrees. Through the control by the drive control unit 67B, the support member 38A that support the plurality of image capturing units 24 rotates in the angular range of more than 180 degrees by using the shaft 38B as the rotation axis. If rotation drive of the support member 38A is started due to the control by the rotation driving unit 28, the image capturing units 24 start to capture images.

For example, the rotation driving unit 28 stops rotation of the support member 38A every time the support member 38A rotates by a predetermined angle. The plurality of image capturing units 24L to 24Q arranged on the support member 38A simultaneously or sequentially capture images of the head portion H on which the mounting member 16 is mounted, every time the rotation of the support member 38A is stopped. Therefore, by repetition of a series of processes including rotation of the support member 38A by the predetermined angle, stop of the rotation of the support member 38A, and image capturing by the image capturing units 24, images of all of the arranged markers 22 are captured.

Further, the drive control unit 67B may further cause the direction driving unit 31 to change the image capturing directions of the image capturing units 24. Therefore, the drive control unit 67B stops the rotation of the support member 38A every time the support member 38A rotates by the predetermined angle. Then, the drive control unit 67B controls the direction driving unit 31 every time the rotation of the support member 38A is stopped. Through the control as described above, it is sufficient for the drive control unit 67B to control the image capturing units 24 and the direction driving unit 31 so as to obtain the captured images 30 in a plurality of different image capturing directions that are achieved by rotation as indicated by D1 and D2 in the figure.

Therefore, it is possible to acquire the captured images 30 of all of the markers 22 arranged on the head portion H.

Consequently, the measurement system 1D of the present embodiment is able to obtain the three-dimensional position information with high accuracy, as the measurement position of the magnetic signal in the measurement target portion of the living body B, similarly to the embodiments as described above.

### Fifth Embodiment

The configuration of the support member that supports the image capturing units 24 is not limited to the embodiments as described above.

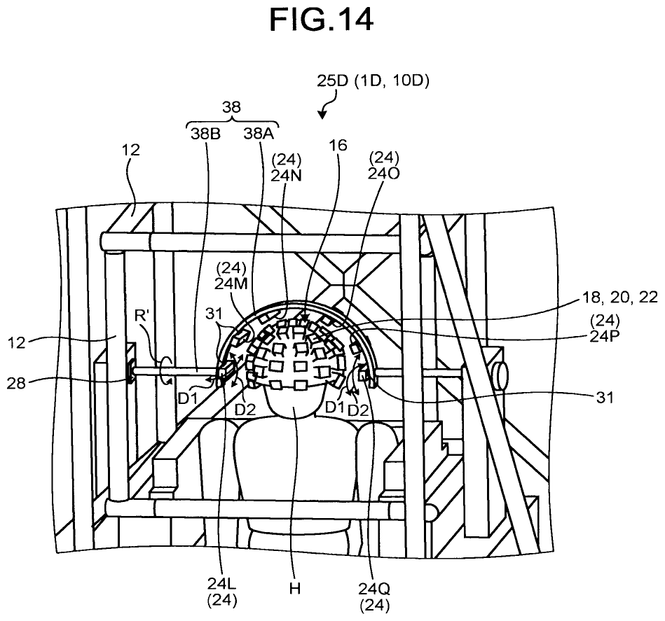

FIG. 15 is a schematic diagram illustrating an example of a moving mechanism unit 25E of a fifth embodiment. The moving mechanism unit 25E is mounted on a measurement apparatus 10E of a measurement system 1E of the present embodiment. Meanwhile, the measurement apparatus 10E of the present embodiment has the same configuration as those of the measurement apparatus 10, the measurement apparatus 10B, the measurement apparatus 10C, and the measurement apparatus 10D as described above except that the measurement apparatus 10E includes the moving mechanism unit 25E instead of the moving mechanism unit 25, the moving mechanism unit 25B, the moving mechanism unit 25C, and the moving mechanism unit 25D of the embodiments as described above.

The moving mechanism unit 25E is a moving mechanism that moves the image capturing units 24 relative to the head portion H of the living body B on which the mounting member 16 is mounted. In the present embodiment, the moving mechanism unit 25E moves the image capturing units 24.

The moving mechanism unit 25E includes a support member 39, the direction driving unit 31, and the movement driving unit 29. The support member 39 includes a support member 39A, a support member 39B, a support member 39C, and a support member 39D.

The support member 39 is a member that supports the image capturing units 24 such that the image capturing units 24 face the head portion H. In other words, the support member 39 supports the image capturing units 24 such that the image capturing units 24 are able to capture images of the markers 22 on the head portion H.

The support member 39A and the support member 39B are arm-shaped members that surround the head portion H, on which the mounting member 16 is mounted, in a front-back direction. The support member 39C and the support member 39D are arm-shaped members that surround the head portion H, on which the mounting member 16 is mounted, in a left-right direction.

An image capturing unit 24R, an image capturing unit 24U, and an image capturing unit 24S, as the image capturing units 24, are mounted on the support member 39C, a support member 39E, and the support member 39D, respectively. The support member 39C, the support member 39D, and the support member 39E are configured with linear members, and the image capturing units 24 are arranged on the respective linear members in one-to-one correspondence. An image capturing unit 24T and the image capturing unit 24U, as the image capturing units 24, are arranged on the support member 39A and the support member 39B, respectively. Each of the support member 39A and the support member 39B is configured with a linear member and another linear member that is perpendicular to the support member 39E, and each of the image capturing unit 24T and the image capturing unit 24U is arranged on one of the two linear members that are arranged in a facing manner.

In the present embodiment, the direction driving unit 31 and the movement driving unit 29 are arranged on each of the image capturing units 24.

FIG. 10 is a functional block diagram of the measurement system 1E of the present embodiment.

The measurement system 1E includes the measurement apparatus 10E and an information processing apparatus 62E. The measurement apparatus 10E and the information processing apparatus 62E are communicably connected to each other. The functional configuration of the measurement apparatus 10E is the same as that of the measurement apparatus 10B. The information processing apparatus 62E is the same as the information processing apparatus 62 except that the information processing apparatus 62E includes a processing unit 69 instead of the processing unit 64. The processing unit 69 is the same as the processing unit 64 except that the processing unit 69 includes a drive control unit 69B instead of the drive control unit 64B.

Explanation will be given below with reference to FIG. 15. The drive control unit 69B controls drive of the direction driving unit 31 and the movement driving unit 29. For example, the drive control unit 69B controls the movement driving unit 29 arranged on each of the image capturing units 24 such that each of the image capturing units 24 moves along an extending direction of the linear member on which each of the image capturing units 24 is arranged. Further, the drive control unit 69B controls the direction driving unit 31 and the image capturing units 24 so as to obtain the captured images 30 in a plurality of different image capturing directions that are archived by rotation as indicated by D1 and D2 in the figure, every time the image capturing units 24 move in a predetermined distance.

Meanwhile, the drive control unit 69B may sequentially move the position of each of the image capturing units 24. Further, the drive control unit 69B may simultaneously move the positions of the plurality of image capturing units 24 and cause the image capturing units 24 to simultaneously capture the captured images 30 every time the image capturing units 24 are moved in the predetermined distance. Furthermore, the drive control unit 69B may control the image capturing units 24 such that the plurality of image capturing units 24 sequentially obtain the captured images every time the image capturing units 24 are moved in the predetermined distance.

Therefore, in the present embodiment, it is possible to acquire the captured images 30 of all of the markers 22 arranged on the head portion H.

Consequently, the measurement system 1E of the present embodiment is able to obtain the three-dimensional position information with high accuracy, as the measurement position of the magnetic signal in the measurement target portion of the living body B, similarly to the embodiments as described above.

Hardware configurations of the information processing apparatus 50 and the information processing apparatus 62 of the embodiments as described above will be described below.

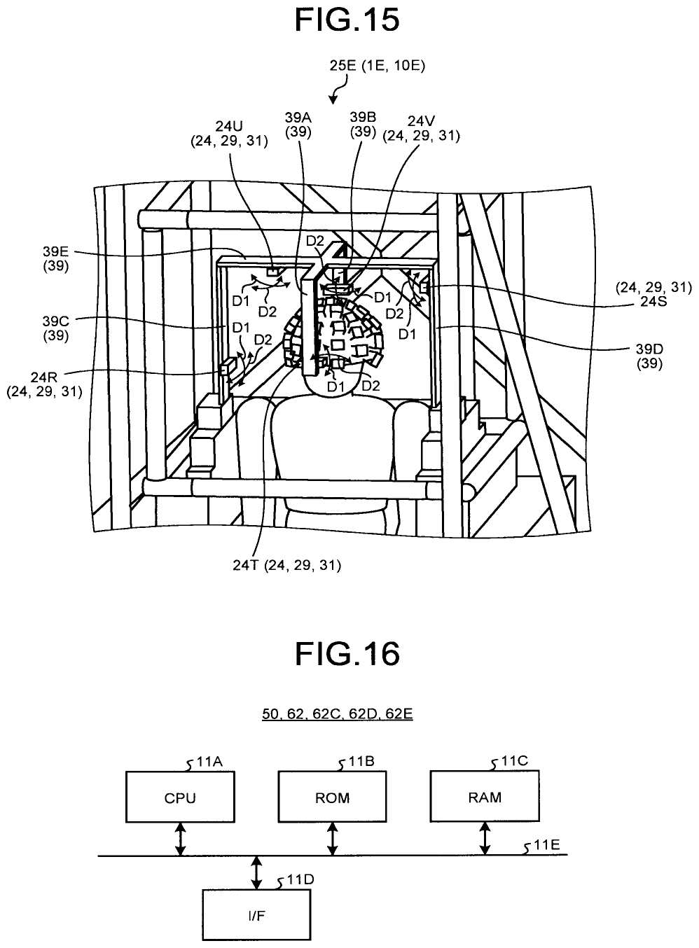

FIG. 16 is a diagram illustrating one example of hardware configurations of the information processing apparatus 50, the information processing apparatus 62, the information processing apparatus 62C, the information processing apparatus 62D, and the information processing apparatus 62E of the embodiments as described above.

Each of the information processing apparatus 50, the information processing apparatus 62, the information processing apparatus 62C, the information processing apparatus 62D, and the information processing apparatus 62E of the embodiments as described above includes a control device, such as a CPU 11A, storage devices, such as a read only memory (ROM) 11B and a random access memory (RAM) 11C, a hard disk drive (HDD), an interface (I/F) 11D that is connected to a network and performs communication, and a bus 11E for connecting each of the units.

A program executed by each of the information processing apparatus 50, the information processing apparatus 62, the information processing apparatus 62C, the information processing apparatus 62D, and the information processing apparatus 62E of the embodiments as described above is provided by being incorporated in the ROM 11B or the like in advance.

The program executed by each of the information processing apparatus 50, the information processing apparatus 62, the information processing apparatus 62C, the information processing apparatus 62D, and the information processing apparatus 62E of the embodiments as described above may be recorded in a computer-readable recording medium, such as a compact disk read only memory (CD-ROM), a flexible disk (FD), a compact disk recordable (CD-R), or a digital versatile disk (DVD), in a computer-installable or computer-executable file format, and may be provided as a computer program product.

Furthermore, the program executed by each of the information processing apparatus 50, the information processing apparatus 62, the information processing apparatus 62C, the information processing apparatus 62D, and the information processing apparatus 62E of the embodiments as described above may be stored in a computer connected to a network, such as the Internet, and may be provided by download via the network. Moreover, the program executed by each of the information processing apparatus 50, the information processing apparatus 62, the information processing apparatus 62C, the information processing apparatus 62D, and the information processing apparatus 62E of the embodiments as described above may be provided or distributed via a network, such as the Internet.

The program executed by each of the information processing apparatus 50, the information processing apparatus 62, the information processing apparatus 62C, the information processing apparatus 62D, and the information processing apparatus 62E of the embodiments as described above may cause a computer to function as each of the information processing apparatus 50, the information processing apparatus 62, the information processing apparatus 62C, the information processing apparatus 62D, and the information processing apparatus 62E as described above. The computer may be implemented by causing the CPU 11A to read the program from the computer-readable storage medium onto a main storage device, and execute the program.

Furthermore, the information processing apparatus 50, the information processing apparatus 62, the information processing apparatus 62C, the information processing apparatus 62D, and the information processing apparatus 62E may be implemented as virtual machines that operate on a cloud computing system.

According to one aspect of the present invention, it is possible to obtain three-dimensional position information with high accuracy, as a measurement position of a magnetic signal in a measurement target portion of a living body.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A measurement system comprising:
a mounting member configured to be mounted on a measurement target portion of a living body and including a plurality of holding units configured to hold a plurality of magnetic sensors such that the plurality of magnetic sensors face the measurement target portion;
an image capturing unit configured to capture images of markers arranged in arrangement regions of the plurality of holding units of the mounting member mounted on the measurement target portion, in the measurement target portion;
a moving mechanism unit configured to move the image capturing unit relative to the measurement target portion on which the mounting member is mounted; and
a derivation unit configured to derive three-dimensional position information on the plurality of magnetic sensors held by the plurality of holding units relative to the measurement target portion, based on the captured images of the markers.

2. The measurement system according to claim 1, wherein the moving mechanism unit includes:
a support member configured to support the image capturing unit; and
at least one of:
a rotation driving unit configured to perform rotation driving of the support member about the measurement target portion as a rotation center; and
a movement driving unit configured to move the support member in a direction along a rotation axis od the rotation driving.

3. The measurement system according to claim 2, wherein an angular range in which the support member is configured to rotate is equal to or larger than 360 degrees.

4. The measurement system according to claim 2, wherein an angular range in which the support member is configured to rotate is equal to or larger than 180 degrees.

5. The measurement system according to claim 2, wherein the support member is supported by the mounting member.

6. The measurement system according to claim 1, wherein each of the plurality of holding units of the mounting member mounted on the measurement target portion has a shape such that at least a part of a marker arranged in an arrangement region in the measurement target portion is not located in a blind area of the image capturing unit.

7. The measurement system according to claim 1, wherein each of the markers arranged on the measurement target portion has a cross-sectional shape conforming to an outer surface of the measurement target portion.

8. The measurement system according to claim 1, wherein the plurality of magnetic sensors comprise a room-temperature magnetic sensor.

9. The measurement system according to claim 8, wherein the plurality of magnetic sensors comprise one of a magnetic resistive sensor and an atomic magnetic sensor.

10. The measurement system according to claim 1, wherein the measurement target portion comprises a head portion of the living body.

11. A measurement apparatus comprising:
a mounting member configured to be mounted on a measurement target portion of a living body and including a plurality of holding units configured to hold a plurality of magnetic sensors such that the plurality of magnetic sensors face the measurement target portion;
an image capturing unit configured to capture images of markers arranged in arrangement regions of the plurality of holding units of the mounting member mounted on the measurement target portion, in the measurement target portion; and
a moving mechanism unit configured to move the image capturing unit relative to the measurement target portion on which the mounting member is mounted.

12. An information processing apparatus comprising:
a derivation unit configured to derive three-dimensional position information on a plurality of magnetic sensors held by a plurality of holding units relative to a measurement target portion of a living body, based on captured images of markers, in a measurement apparatus,
the measurement apparatus including:
a mounting member configured to be mounted on the measurement target portion and including the plurality of holding units configured to hold the plurality of magnetic sensors such that the plurality of magnetic sensors face the measurement target portion;
an image capturing unit that captures the images of the markers arranged in arrangement regions of the plurality of holding units of the mounting member mounted on the measurement target portion, in the measurement target portion; and
a moving mechanism unit configured to move the image capturing unit relative to the measurement target portion on which the mounting member is mounted.
